(19) United States
(12) Patent Application Publication
(10) Pub. No.: US 2025/0213132 A1
(43) Pub. Date: Jul. 3, 2025

(54) MACHINE LEARNING DETECTION OF HYPERMETABOLIC CANCER BASED ON NUCLEAR MAGNETIC RESONANCE SPECTRA

(71) Applicant: THE JOHNS HOPKINS UNIVERSITY, Baltimore, MD (US)

(72) Inventors: Meiyappan SOLAIYAPPAN, Ellicott City, MD (US); Santosh Kumar BHARTI, Uttar Pradesh (IN); Zaver M. BHUJWALLA, Baltimore, MD (US); Michael GOGGINS, Baltimore, MD (US); Malcolm V. BROCK, Baltimore, MD (US)

(21) Appl. No.: 18/853,196

(22) PCT Filed: Apr. 7, 2023
(86) PCT No.: PCT/US2023/017844

§ 371 Date: Oct. 1, 2024

Related U.S. Application Data
(60) Provisional application No. 63/329,113, filed on Apr. 8, 2022.

(51) Int. Cl. A61B 5/055; A61B 5/00; G01N 33/574; G16H 50/20

(52) U.S. Cl. CPC A61B 5/055; A61B 5/7264; G01N 33/57488; G16H 50/20

(57) ABSTRACT

A computer-implemented machine learning system for, and method of, detecting a hypermetabolic cancer based on a nuclear magnetic resonance spectrum of a patient biofluid is presented. The techniques includes obtaining a nuclear magnetic resonance spectrum of a patient biofluid; providing the nuclear magnetic resonance spectrum to a machine learning system trained with a training corpus, the training corpus including a group of normal biofluid nuclear magnetic resonance spectra and a group of hypermetabolic cancer biofluid nuclear magnetic resonance spectra; and supplying an indication based on an output of the machine learning system, where the indication is representative of whether the nuclear magnetic resonance spectrum of the patient biofluid is indicative of cancer.

### CROSS-REFERENCE TO RELATED APPLICATIONS

This application is the national stage entry of International Patent Application No. PCT/US2023/017844, filed on Apr. 7, 2023, and published as WO 2023/196571 A1 on Oct. 12, 2023, which claims the benefit of U.S. Provisional Patent Application No. 63/329,113, filed on Apr. 8, 2022, which are hereby incorporated by reference herein in their entireties.

### GOVERNMENT SUPPORT

This invention was made with government support under grant CA209960 awarded by the National Institute of Health. The government has certain rights in the invention.

### FIELD

This disclosure relates generally to cancer detection.

### BACKGROUND

The ready availability and easy accessibility of blood has resulted in blood-based analytes being used for a range of clinical diagnostic tests. Plasma and serum samples are attractive because the standardization of sample collection, such as fasting times and storage, supports reproducibility of the measurements. Blood-based tests have, however, have met with limited success in detecting cancer, other than in a handful of cancer types such as prostate and ovarian cancer. The detection of circulating tumor cells and DNA in liquid biopsies has attracted significant interest, but challenges of sensitivity and specificity remain. For example, assessment of circulating proteins and mutations in cell-free DNA was found to detect eight cancer types, including pancreas and lung cancer, with a median sensitivity of 70% and a specificity of 99%. See Cohen, J.D., et al., Detection and localization of surgically resectable cancers with a multi-analyte blood test, Science 359, 926-930 (2018). Proteomic-based technologies such as liquid chromatography mass spectrometry are attracting significant interest for the purpose of cancer detection, but have challenges such as standardization of sample preparation and processing, and quantitation. See Bhawal, R. et al., Challenges and Opportunities in Clinical Applications of Blood-Based Proteomics in Cancer, Cancers (Basel), 12 (2020).

Pancreatic ductal adenocarcinoma (“PDAC”) is the most frequent form of pancreatic cancer, and its dismal survival rate of less than 10% at five years makes it the fourth leading cause of cancer-related deaths. The poor prognosis of PDAC is mainly due to late-stage diagnosis. Only 20% of pancreatic cancers are resectable by the time they are detected. Similarities in the clinical behavior and imaging features of PDAC and chronic pancreatitis further complicate the detection of PDAC.

Lung cancer is the most common cause of cancer death world-wide. Approximately 85% of all lung cancers are non-small cell lung cancer (“NSCLC”). The presence of metastatic disease at the time of diagnosis in most patients is a major cause of lung cancer mortality, highlighting the importance of early detection and screening. Important advances have been made in the treatment of NSCLC, but overall cure and survival rates remain low especially with advanced disease. Although low-dose computer tomography (“CT”) is available for lung cancer screening, it is recommended only for adults who are at high risk for developing the disease because of their smoking history and age. CT imaging results in exposure to radiation. Additionally, according to the American Cancer Society, 20% of individuals who succumbed to lung cancer were non-smokers, highlighting the importance of lung cancer screening in larger populations.

Applications of artificial intelligence (“AI”) in the cancer imaging space have largely focused on detecting lung and pancreatic cancer from conventional radiology images. However, the high prevalence of lung nodules and pancreatic cysts in the general population makes it challenging to predict the likelihood of cancer from an incidental finding. Even with 99% sensitivity and specificity, the remaining 1% represents a large number of patients who may need to undergo high-risk surgical procedures. The subtle CT features of early PDAC can lead to a missed diagnosis. In the case of lung cancer, AI tools for early detection using CT scans have been developed using the National Lung Screening Trial datasets. These low-dose CT data, collected from high-risk populations, were used to demonstrate that AI techniques can perform on par with radiologists, thus providing an effective fully automated screening tool. While AI techniques based on routine single modality clinical imaging methods such as CT or magnetic resonance imaging (“MRI”) have provided accuracies that were previously unattainable, the solutions still fail to provide cancer screening that is non-invasive, simple to measure, cost-effective, radiation-free, and rapid to provide results.

Nuclear magnetic resonance (“NMR”) spectroscopy is a spectroscopic technique that can be used to detect individual organic compounds in a chemical sample. The sample is exposed to a strong magnetic field and radio waves, and a nuclear magnetic resonance signal is produced, which is indicative of chemical compounds in the sample. An example of NMR spectroscopy is high-resolution proton NMR spectroscopy, or ¹H magnetic resonance spectroscopy, which relies on the nuclear magnetic resonance of hydrogen-1 nuclei.

### SUMMARY

According to various method embodiments, a computer implemented machine learning method of detecting a hypermetabolic cancer based on a nuclear magnetic resonance spectrum of a patient biofluid is presented. The method includes: obtaining a nuclear magnetic resonance spectrum of a patient biofluid; providing the nuclear magnetic resonance spectrum to a machine learning system trained with a training corpus, the training corpus including a group of normal biofluid nuclear magnetic resonance spectra and a group of hypermetabolic cancer biofluid nuclear magnetic resonance spectra; and supplying an indication based on an output of the machine learning system, where the indication is representative of whether the nuclear magnetic resonance spectrum of the patient biofluid is indicative of cancer.

Various optional features of the above method embodiments include the following. The method may further include providing clinical follow-up for the patient upon an indication of cancer. The obtaining may include obtaining a ¹H nuclear magnetic resonance spectrum of the patient biofluid. The obtaining may include obtaining a pre-saturation and single pulse sequence nuclear magnetic resonance spectrum of the patient biofluid. The group of hypermetabolic cancer biofluid nuclear magnetic resonance spectra may include pancreatic ductal adenocarcinoma biofluid nuclear magnetic resonance spectra, and the indication may be representative of whether the nuclear magnetic resonance spectrum of the patient biofluid is indicative of pancreatic ductal adenocarcinoma. The group of hypermetabolic cancer biofluid nuclear magnetic resonance spectra may include non-small cell lung cancer biofluid nuclear magnetic resonance spectra, and the indication may be representative of whether the nuclear magnetic resonance spectrum of the patient biofluid is indicative of non-small cell lung cancer. The providing the nuclear magnetic resonance spectrum may include providing at least 30,000 nuclear magnetic resonance spectrum data points. The providing the nuclear magnetic resonance spectrum may include providing nuclear magnetic resonance spectrum data points that substantially cover a range of 10 ppm to 0.5 ppm. The providing the nuclear magnetic resonance spectrum may include providing nuclear magnetic resonance spectrum data points that cover the range of 10 ppm to 0.5 ppm, excluding a solute and any contaminant. The providing the nuclear magnetic resonance spectrum may include providing nuclear magnetic resonance spectrum data points representing for at least regions for: lipid (0.9 ppm), BCAA, lipid (1.2 ppm), lipid (1.6 ppm), acetate, lipid (2.03 ppm), glutamine, lactate, glucose, myo-inositol, and betahydroxybutyrate. The providing the nuclear magnetic resonance spectrum may include providing nuclear magnetic resonance spectrum data points representing for at least regions for: lipid (0.9 ppm), leucine, isoleucine, valine, BCAA (leucine+isoleucine+valine), lipid (1.2 ppm), alanine, lipid (1.6 ppm), acetate, lipid (2.03 ppm), acetone, acetoacetate, pyruvate, glutamate, glutamine, creatine, phosphocreatine, lactate, glucose, PUFA, tyrosine, histidine, phenylalanine, 1.17 ppm, citrate, myo-inositol, 4.14 ppm, betahydroxybutyrate, and glutamine/glutamate. The method may further include the machine learning system deriving a feature vector from the nuclear magnetic resonance spectrum, the feature vector including at least 3000 entries. The patient biofluid may include one of blood serum or blood plasma. The machine learning system may include an artificial neural network. The training corpus may further include a group of benign disease biofluid nuclear magnetic resonance spectra.

According to various system embodiments, a computer system is presented. The computer system includes an electronic processor and computer-readable instructions that, when executed by the electronic processor, configure the electronic processor to perform actions including the actions of any of the method embodiments described herein.

According to various computer readable medium embodiments, a non-transitory computer readable medium is presented. The non-transitory computer readable medium includes instructions that, when executed by an electronic processor, configure the electronic processor to perform the actions of any of the method embodiments described herein.

According to various embodiments, a computer implemented machine learning method of detecting a hypermetabolic cancer based on a nuclear magnetic resonance spectrum of a patient biofluid is presented. The method includes obtaining a nuclear magnetic resonance spectrum of a patient biofluid; providing the nuclear magnetic resonance spectrum to a machine learning system trained with a training corpus, the training corpus including a group of normal biofluid nuclear magnetic resonance spectra and a group of hypermetabolic cancer biofluid nuclear magnetic resonance spectra; and supplying an indication based on an output of the machine learning system, where the indication is representative of whether the nuclear magnetic resonance spectrum of the patient biofluid is indicative of cancer.

Various optional features of the above method include the following. The method may include providing clinical follow-up for the patient upon an indication of cancer. The group of hypermetabolic cancer biofluid nuclear magnetic resonance spectra may include pancreatic ductal adenocarcinoma biofluid nuclear magnetic resonance spectra, and the indication may be representative of whether the nuclear magnetic resonance spectrum of the patient biofluid is indicative of pancreatic ductal adenocarcinoma. The group of hypermetabolic cancer biofluid nuclear magnetic resonance spectra may include non-small cell lung cancer biofluid nuclear magnetic resonance spectra, and the indication may be representative of whether the nuclear magnetic resonance spectrum of the patient biofluid is indicative of non-small cell lung cancer. The training corpus may include at least one spectrum from a sample determined to be a pivot sample. The providing the nuclear magnetic resonance spectrum may include providing nuclear magnetic resonance spectrum data points that cover a range of 10 ppm to 0.5 ppm, excluding a solute and any contaminant. The providing the nuclear magnetic resonance spectrum may include providing nuclear magnetic resonance spectrum data points representing for at least regions for: lipid (0.9 ppm), BCAA, lipid (1.2 ppm), lipid (1.6 ppm), acetate, lipid (2.03 ppm), glutamine, lactate, glucose, myo-inositol, and betahydroxybutyrate. The machine learning system may be trained to output a classification of the nuclear magnetic resonance spectrum into one of a plurality of classes, and the method may further include deriving a respective feature vector from the nuclear magnetic resonance spectrum for each pair of classes of the plurality of classes. Each respective feature vector may encode differences between the nuclear magnetic resonance spectrum and a spectrum representing a respective base class, where the differences are determined at each of a plurality of spectral regions. The training corpus may further include a group of benign disease biofluid nuclear magnetic resonance spectra.

According to various embodiments, a system for detecting a hypermetabolic cancer based on a nuclear magnetic resonance spectrum of a patient biofluid is presented. The system includes: a machine learning system trained with a training corpus, the training corpus including a group of normal biofluid nuclear magnetic resonance spectra and a group of hypermetabolic cancer biofluid nuclear magnetic resonance spectra; an electronic processor; and a non-transitory computer-readable medium communicatively coupled to the electronic processor and including instructions that, when executed by the electronic processor, configure the electronic processor to perform actions including: obtaining a nuclear magnetic resonance spectrum of a patient biofluid; providing the nuclear magnetic resonance spectrum to the machine learning system; and supplying an indication based on an output of the machine learning system, where the indication is representative of whether the nuclear magnetic resonance spectrum of the patient biofluid is indicative of cancer.

Various optional features of the above system include the following. The system may further include a nuclear magnetic resonance spectrometer, where the obtaining includes obtaining the nuclear magnetic resonance spectrum of the patient biofluid from the nuclear magnetic resonance spectrometer. The group of hypermetabolic cancer biofluid nuclear magnetic resonance spectra may include pancreatic ductal adenocarcinoma biofluid nuclear magnetic resonance spectra, and the indication may be representative of whether the nuclear magnetic resonance spectrum of the patient biofluid is indicative of pancreatic ductal adenocarcinoma. The group of hypermetabolic cancer biofluid nuclear magnetic resonance spectra may include non-small cell lung cancer biofluid nuclear magnetic resonance spectra, and the indication may be representative of whether the nuclear magnetic resonance spectrum of the patient biofluid is indicative of non-small cell lung cancer. The training corpus may include at least one spectrum from a sample determined to be a pivot sample. The providing the nuclear magnetic resonance spectrum may include providing nuclear magnetic resonance spectrum data points that cover a range of 10 ppm to 0.5 ppm, excluding a solute and any contaminant. The providing the nuclear magnetic resonance spectrum may include providing nuclear magnetic resonance spectrum data points representing for at least regions for: lipid (0.9 ppm), BCAA, lipid (1.2 ppm), lipid (1.6 ppm), acetate, lipid (2.03 ppm), glutamine, lactate, glucose, myo-inositol, and betahydroxybutyrate. The machine learning system may be trained to output a classification of the nuclear magnetic resonance spectrum into one of a plurality of classes, and the actions may further include deriving a respective feature vector from the nuclear magnetic resonance spectrum for each pair of classes of the plurality of classes. Each respective feature vector may encode differences between the nuclear magnetic resonance spectrum and a spectrum representing a respective base class, where the differences are determined at each of a plurality of spectral regions. The training corpus may further include a group of benign disease biofluid nuclear magnetic resonance spectra.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

The above and/or other aspects and advantages will become more apparent and more readily appreciated from the following detailed description of examples, taken in conjunction with the accompanying drawings, in which:

FIG. 1 depicts nuclear magnetic resonance (“NMR”) spectra of blood plasma from normal individuals, individuals with pancreatic ductal adenocarcinoma, (“PDAC”), and individuals with benign pancreatic disease, as used for the first reductions to practice;

FIG. 2 depicts NMR spectra of blood serum from individuals with non-small cell lung cancer, and individuals with benign lung disease, as used for the first reductions to practice;

FIG. 3 depicts a table summarizing human participant data used for the first reductions to practice described herein;

FIG. 4 depicts charts that analyze results produced by a first reduction to practice for NSCLC using Carr-Purcell-Meiboom-Gill (“CPMG”) spectra;

FIG. 5 depicts charts that analyze results produced by a first reduction to practice for non-small cell lung cancer (“NSCLC”) using single-pulse water suppression by pre-saturation (“ZGPR”) spectra;

FIG. 6 depicts a scatter plot showing classification variables according to the first reductions to practice;

FIG. 7 depicts a table showing differences in individual metabolites detected from CPMG NMR spectra according to a first reduction to practice;

FIG. 8 depicts scatter plots for principal component analysis of normal, benign pancreatic disease, and PDAC NMR spectra;

FIG. 9 depicts partial least squares loadings and scatter plots for supervised partial least squares regression analysis of normal, benign pancreatic disease, and PDAC NMR spectra;

FIG. 10 depicts discrimination-determining CPMG spectral regions identified by an analysis according to a first reduction to practice;

FIG. 11 depicts discrimination-determining ZGPR spectral regions identified by an analysis according to a first reduction to practice;

FIG. 12 is a schematic diagram of a machine learning system, including a single-channel artificial neural network, according to the first reductions to practice;

FIG. 13 is a schematic diagram of a machine learning system, including a two-channel artificial neural network, according to an example embodiment;

FIG. 14 is a schematic diagram of a machine learning system, including a three-channel artificial neural network, according to a second reduction to practice;

FIG. 15 depicts a method of spectral region of interest determination according to the second reduction to practice;

FIG. 16 depicts positive and negative spectral region of interest groupings as used in the second reduction to practice;

FIG. 17 schematically illustrates a method of neural network training as used for the second reduction to practice;

FIG. 18 schematically illustrates the method of inference for classifying a new sample spectrum as used for the second reduction to practice;

FIG. 19 shows a confusion matrix corresponding to the training phase validation of the second reduction to practice; and

FIG. 20 shows confusion matrices corresponding to an inference phase validation of the second reduction to practice using blinded test samples.

### DETAILED DESCRIPTION

Embodiments as described herein are described in sufficient detail to enable those skilled in the art to practice the invention and it is to be understood that other embodiments may be utilized and that changes may be made without departing from the scope of the invention. The present description is, therefore, merely exemplary.

Some embodiments utilize machine learning, e.g., by way of an artificial neural-network, to detect pancreatic ductal adenocarcinoma (“PDAC”) and/or non-small cell lung cancer (“NSCLC”) from nuclear magnetic resonance (“NMR”) spectra, such as ¹H NMR spectra, of circulating metabolites from a human biofluid sample, e.g., blood plasma and/or blood serum. Some embodiments discriminate between patients with no clinical evidence of pancreatic, lung, or other organ disease, individuals with benign pancreatic, lung, or other organ disease, and individuals with PDAC, NSCLC, or other hypermetabolic cancer. The aberrant metabolism of hypermetabolic cancer in general, and PDAC and NSCLC, in particular, are reflected as changes in circulating metabolites. Some embodiments use artificial neural networks to map the pattern of subtle changes in ¹H NMR spectra input data to the corresponding disease groups or classes with a high degree of sensitivity and specificity.

Some embodiments analyze substantially the entire NMR spectral range, e.g., the entire NMR spectral range excluding a solute and/or any contaminants.

Some embodiments meet the need for a cancer test, e.g., for routine screening, that provides for non-invasiveness, ease of measurement, cost-effectiveness, radiation-freedom, and rapid results.

Several reductions to practice, described throughout this disclosure, provided high degrees of sensitivity and specificity. The reductions to practice utilized spectra of blood plasma obtained from individuals with no evidence of pancreatic or lung disease, benign pancreatic or lung disease, and PDAC or NSCLC, using ¹H NMR spectra obtained using both the Carr-Purcell-Meiboom-Gill (“CPMG”) sequence that results in spectra with a flat baseline, and using a single pulse sequence with water pre-saturation (“ZGPR”) that produced broad resonances in the baseline. The reductions to practice used machine learning, including artificial neural networks, to classify blood plasma spectra as indicative of normal, non-malignant disease, or malignant disease.

In sum, as described in detail herein, some embodiments provide an accurate, robust, rapid, radiation-free, and cost-effective biofluid-based artificial intelligence system to detect and screen for PDAC, NSCLC and other hypermetabolic cancers.

### I. Description of the First Reductions to Practice

The first reductions to practice described herein included multiple individual reductions to practice, each of which utilizes a one-channel artificial neural network architecture. The first reductions to practice included a reduction to practice that was trained to classify a blood plasma CPMG NMR spectrum into normal, benign pancreatic disease, or PDAC. The first reductions to practice also included a reduction to practice that was trained to classify a blood plasma ZPGR NMR spectrum into normal, benign lung/pancreatic disease, or NSCLC. The first reductions to practice were validated by comparing results with those produced using multivariate pattern recognition (e.g., principal component analysis and partial least-squares regression).

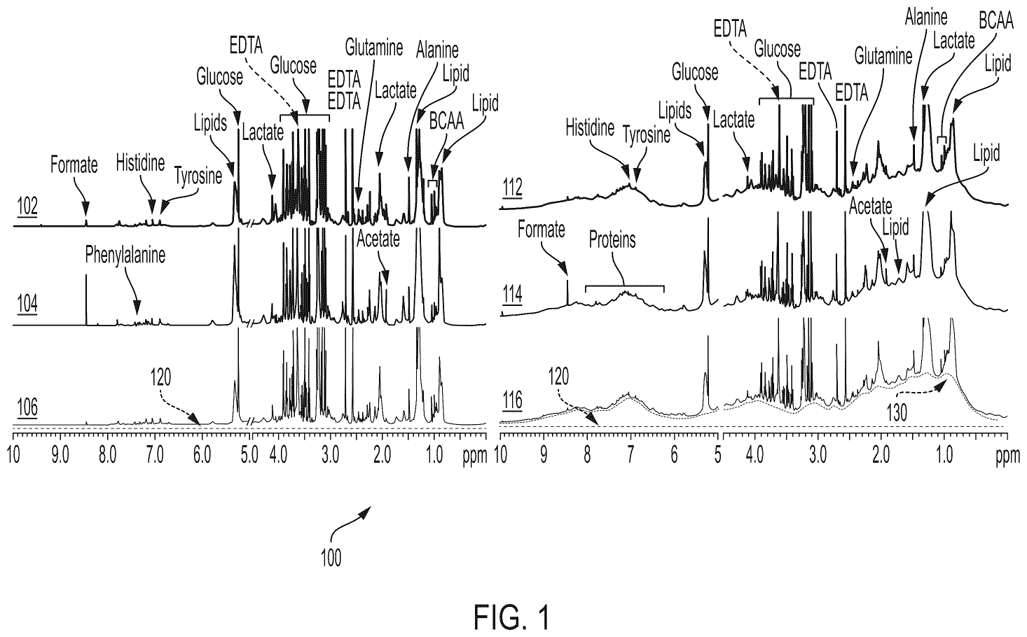

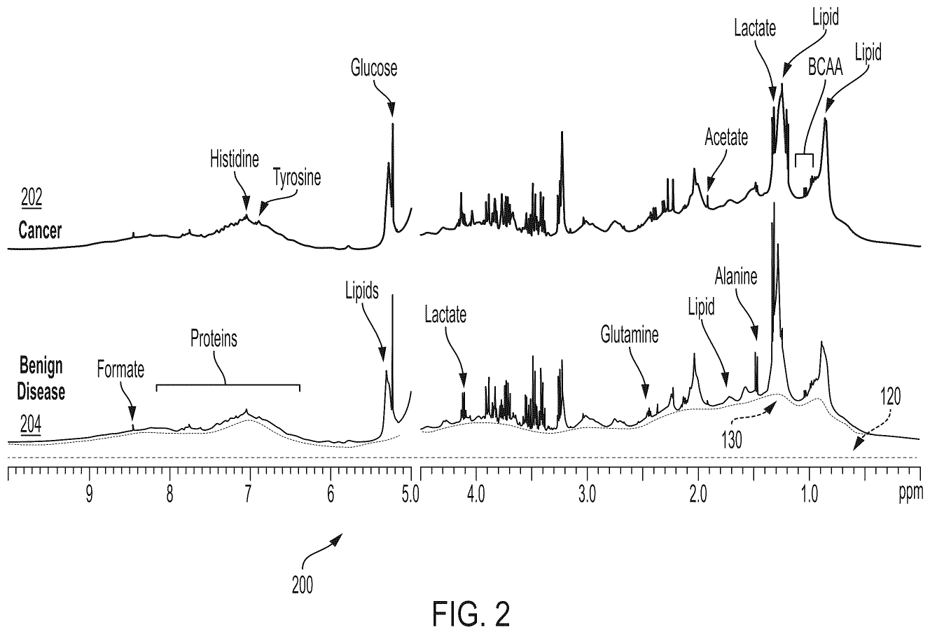

FIG. 1 depicts NMR spectra 100 of blood plasma from normal individuals 106, 116 individuals with PDAC 104, 114, and individuals with benign pancreatic disease 102, 112 as used for the first reductions to practice. FIG. 2 depicts NMR spectra 200 of blood serum from individuals with non-small cell lung cancer 202, and individuals with benign lung disease 204, as used for the first reductions to practice. Spectra 102, 104, 106 were acquired using a CPMG pulse sequence (short T2 filtering) with water presaturation to suppress the broad resonances from lipoproteins/albumins. Spectra 112, 114, 116, 202, 204, were acquired using a single pulse ZGPR sequence with water presaturation. The ZGPR ¹H NMR spectra 112, 114, 116, 202, 204 retained the broad resonances from macromolecules such as lipids, lipoproteins/albumins, unlike the CPMG spectra 102, 104, 106 that provided a flatter baseline due to suppression of broad signals using short T2 filtering. Spectral regions from 5.0-10.0 ppm were vertically magnified at 2× to better visualize the low intensity peaks in those regions. Dotted lines 120 indicate the broad peaks that arise from macromolecules, e.g., lipids and lipoproteins/albumins present in the plasma. Dotted lines 130 identify the flat baseline in CPMG spectra that primarily detects signal from small molecules. In FIGS. 1 and 2, BCAA refers to branch chain amino acids, and the detected EDTA represents a contaminant from blood collection tubes.

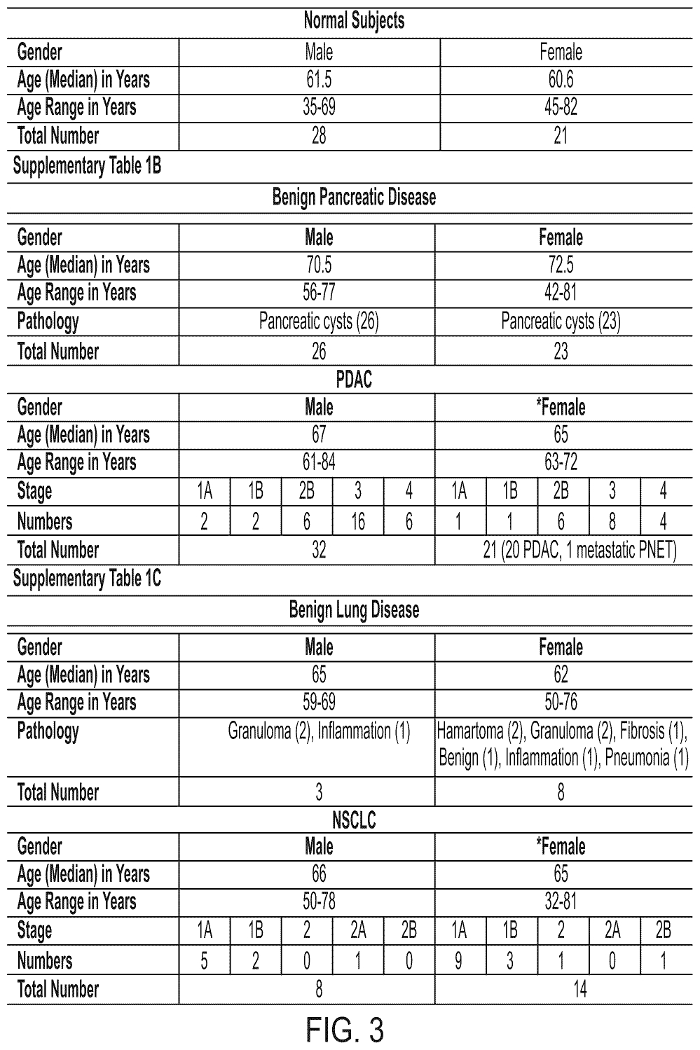

FIG. 3 depicts a table 300 summarizing human participant data used for the first reductions to practice described herein. As shown, Table 1 includes age, gender, and disease stage for normal individuals, pancreatic group individuals, and lung group individuals. Of the pancreatic group individuals, one female was subsequently diagnosed with metastatic neuroendocrine pancreatic cancer instead of PDAC.

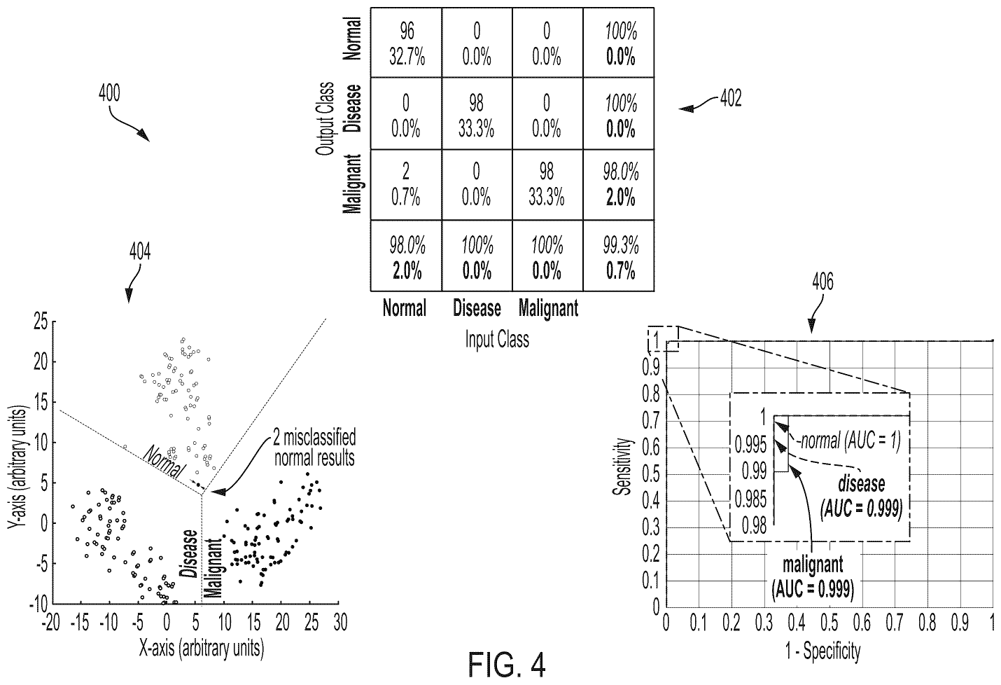

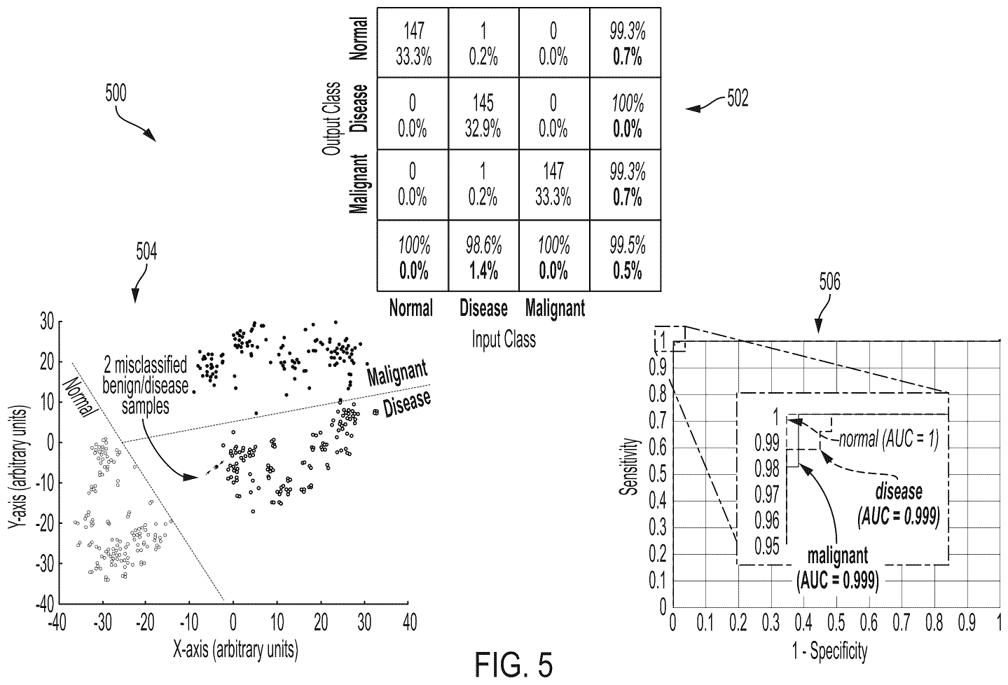

Example results of the neural network analyses of the first reductions to practice are shown and described presently in reference to FIGS. 4 and 5.

FIG. 4 depicts charts 400 that analyze results produced by a first reduction to practice for PDAC using CPGM spectra. In particular, FIG. 4 depicts confusion matrix 402, scatter plot 404, and receiver operating characteristic (“ROC”) curves 406.

The discrimination of the pancreatic group based on the CPMG plasma spectra is presented as a confusion matrix 402. The classification accuracy was 100% for the benign disease and malignant groups, and 98% for the normal group. The diagonal boxes, identified with diagonal-line shading, show the correct predictions in each class, and boxes identified with cross-hatch shading indicate misclassifications. The numbers in each box correspond to the number of samples (and their percentage of the total data). The column at the far right shows the precision value (positive predictive value) for each predicted class (top numbers). The bottom-row shows the prediction accuracy value for each class (top numbers) and the bottom-right corner box shows the overall accuracy value (top number) and error rate (bottom number). Cancer classification resulted in a 99.5% correct prediction.

The scatter plot 404 shows the 2D embedding of the neural network's classification variables to illustrate the effective classification of normal, disease, and malignant (here, PDAC) samples with just two samples misclassified. The scatter plot 404 demonstrates the clear separation between the normal, benign disease and malignant groups for PDAC. The two normal samples misclassified as malignant (false positives) are shown near the center of the scatter plot 304 within the normal cluster.

The ROC curves 406 show the sensitivity and specificity performance of the neural-network, with the area under curve (“AUC”) for all three classifications above 0.999. Although the accuracy of discrimination for normal cases is 98% in the confusion matrix 402, the corresponding AUC number is 1.0 because the two misclassified normal cases had almost equal probability of being classified as either normal or malignant, with the malignant probability being only slightly higher. This resulted in the two cases being misclassified as malignant, while the ROC curve that is based on binary classification of the probability numbers resulted in a higher discrimination measure.

FIG. 5 depicts charts 500 that analyze results produced by a first reduction to practice for NSCLC and PDAC using single-pulse water suppression by ZGPR spectra. In particular, FIG. 5 depicts confusion matrix 502, scatter plot 504, and ROC curves 506. Charts 500 represent ZGPR spectra from combined pancreatic and lung groups.

The confusion matrix 502 shows the result of PDAC and NSCLC prediction using plasma and serum ZGPR spectra. The diagonal boxes, shaded using diagonal lines, show the correct predictions in each class and boxes shaded with cross-hatching indicate misclassifications. The numbers in each box correspond to the number of samples (and their percentage of the total data). The column at the far right shows the precision value (positive predictive value) for each predicted class (top numbers). The bottom row shows the prediction accuracy value for each class (italicized top numbers) and the bottom-right corner box shows the overall accuracy value (top number) and error rate (bottom number). The classification accuracy was 100% for the normal and malignant groups, and 98.6% for the benign disease group. Cancer classification resulted in a 99.5% correct prediction.

The scatter plot 504 shows the 2D embedding of the neural-network's classification variables to illustrate the effective classification of normal, disease, and malignant (PDAC, NSCLC) samples with just two samples misclassified. Specifically, two samples belonging to the benign disease group were misclassified as normal (false negative) and malignant (false positive) in the scatter plot 504.

The ROC curves 506 show the sensitivity and specificity performance of the neural network of a first reduction to practice, with the AUC for all three classifications above 0.999, indicating a 99.9% classification performance. The ROC curves were based on the binary classification of the probability of detection of each class. The confusion matrix 502 results were based on the comparison of the probability numbers across the three classes with the highest probability determining which class the sample belongs to. When the highest and the next highest probability number are high and very close, the confusion matrix will pick the highest probability for assignment resulting in misclassification, but the probability will still be high enough for the AUC value to not be impacted. On the other hand, if the probability numbers are low but very close, the confusion matrix can still assign the correct class but because the probability number is low, the AUC value will decrease.

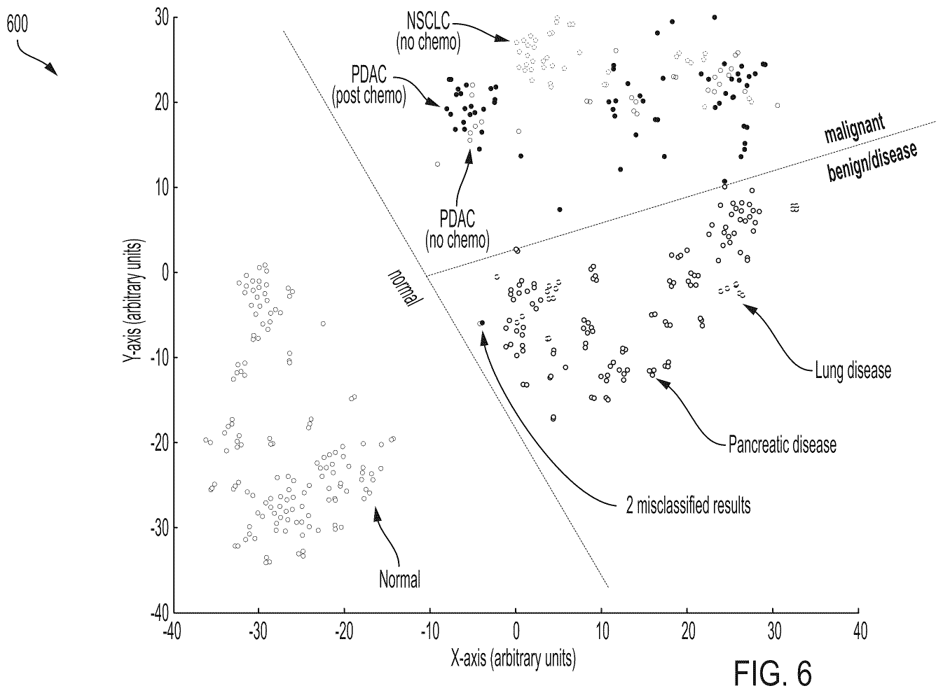

FIG. 6 depicts a scatter plot 600 showing classification variables according to the first reductions to practice. In particular, scatter plot 600 analyzes the classifications of the groups to determine the effects, if any, of using a different field strength or serum samples for spectra obtained from NSCLC and benign lung disease individuals. Thus, scatter plot 600 shows the 2D embedded neural network's classification variables with the NSLC, benign lung disease, and PDAC with and without chemotherapy color coded. As shown in the scatter plot 600, these groups did not cluster together indicating no significant influence of field strength or the use of serum instead of plasma samples. Although plasma samples from PDAC patients were obtained at least one month after the end of chemotherapy treatment, scatter plot 600 evaluates if the treatment influenced classification. Serum samples from NSCLC individuals were treatment naïve. As shown in scatter plot 600, there was no clear separation between the treated and untreated samples.

### II. Validation of the First Reductions to Practice

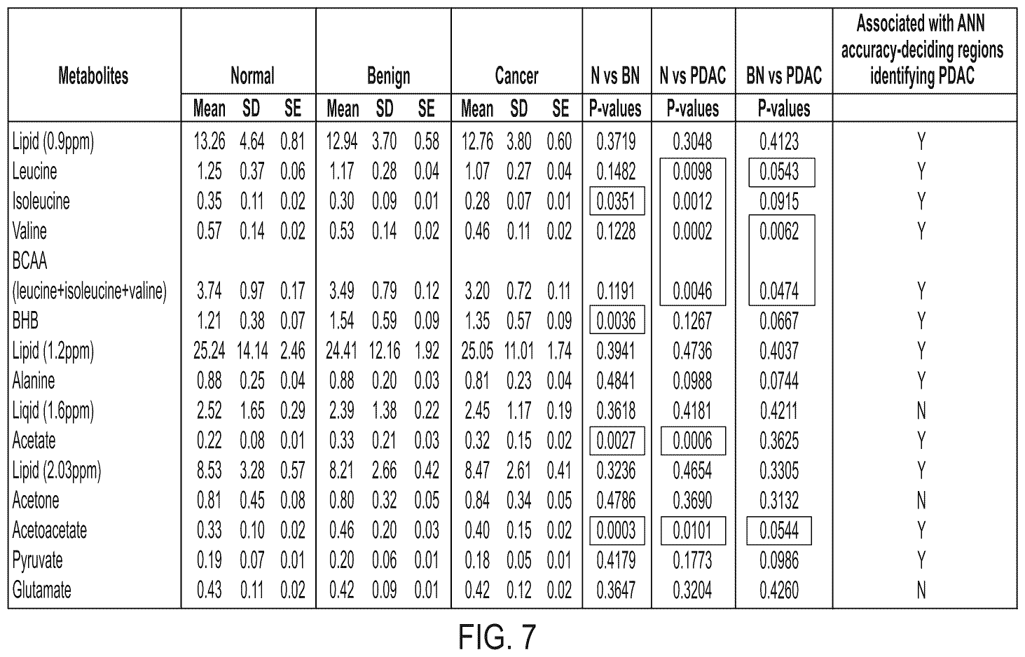

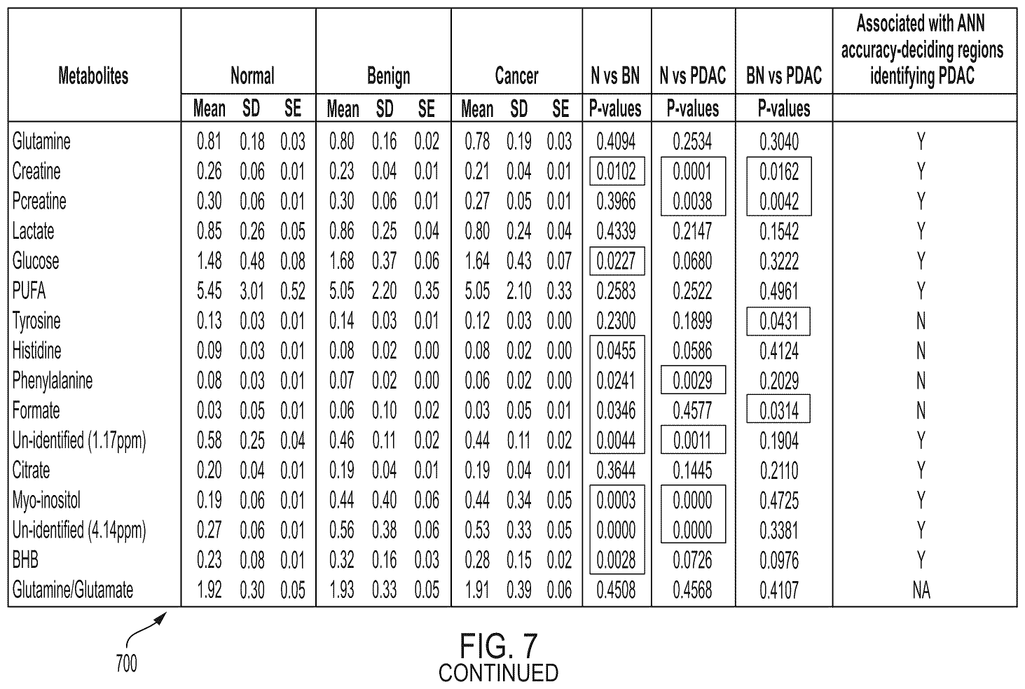

This section develops and compares, with favorable results, non-machine learning classification approaches to the machine learning approach of the first reductions to practice. In particular, this section presents principal component analysis (“PCA”) and partial least square regression (“PLS”) analyses to evaluate plasma spectral patterns and whether each group (normal, benign pancreatic disease and PDAC) could be specifically defined by overall spectral patterns obtained from CPMG or ZGPR spectra using multivariate pattern recognition analysis. Using Bruker AMIX software, an equal-size (0.01 ppm) binning method was used to digitize the plasma spectra into multiple bins with a 0.01 ppm width. Water and EDTA resonances were excluded from the analysis. Integral peak areas were normalized to the reference peak as well as the plasma sample volume. Data from a univariate analysis summarizing differences in individual metabolites detected from the CPMG spectra are presented in FIG. 7.

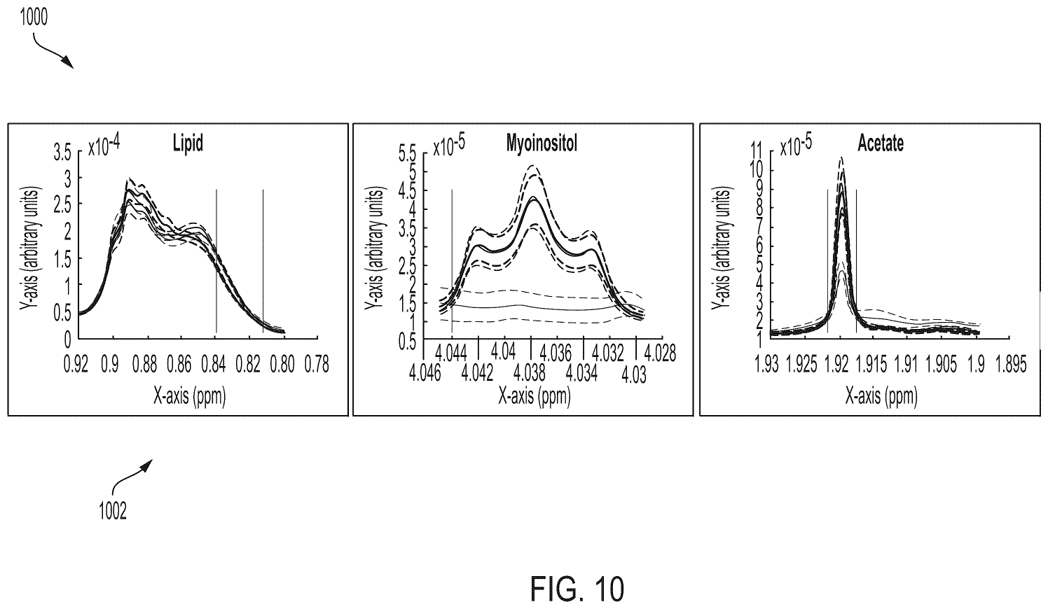

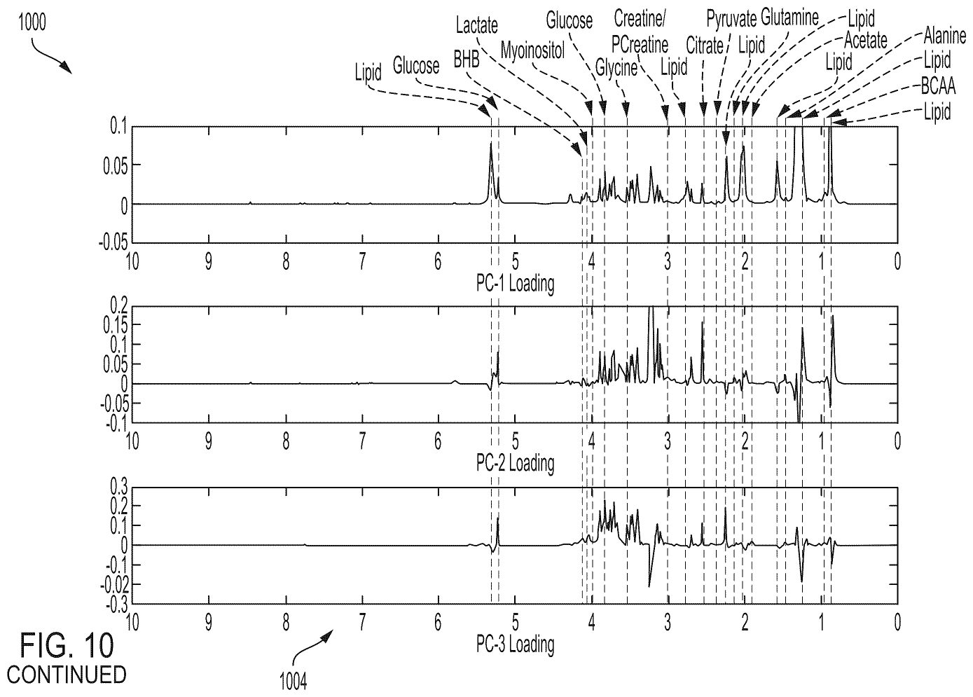

FIG. 7 depicts a table 700 showing differences in individual metabolites detected from CPMG NMR spectra according to a first reduction to practice. In the table 700, the mean, standard deviation, and standard error are shown for plasma metabolites quantified by ¹H NMR CPMG spectroscopy for normal, benign pancreatic disease and PDAC groups. Metabolites that significantly differed between normal, benign pancreatic disease and PDAC groups, along with their associated magnitude of change, are highlighted. P-values<0.05 were considered significant. Although significant differences in some of the metabolites are apparent from the univariate analysis, the analysis of spectral patterns in their entirety by the first reductions to practice classified the three groups with a high confidence that was not readily apparent with the univariate analysis. The rightmost column of the table 700 is described below in reference to the results of FIG. 10.

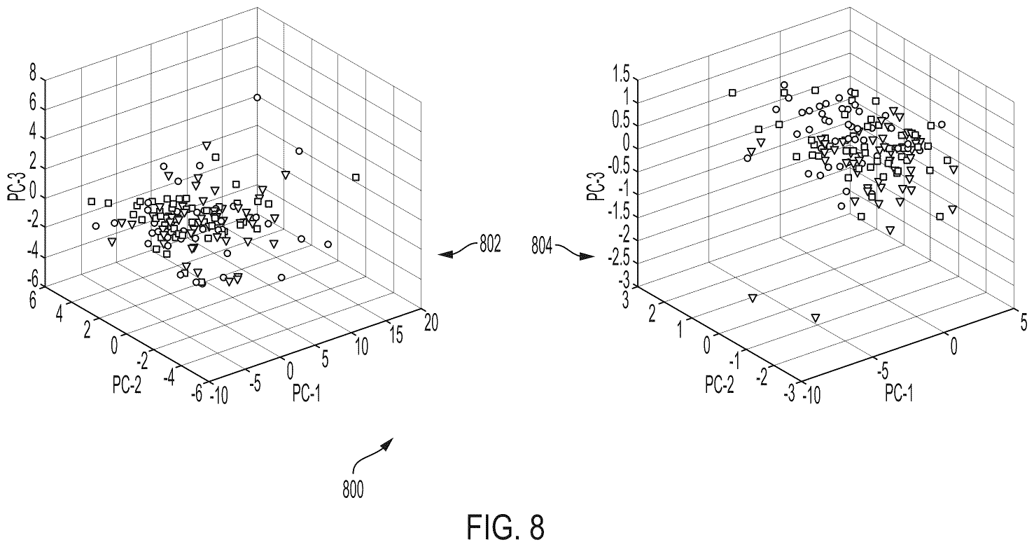

FIG. 8 depicts scatter plots 800 for PCA of normal, benign pancreatic disease, and PDAC NMR spectra. In particular, FIG. 8 shows score plots derived from PCA of spectra from plasma of normal (circle), pancreatic disease (square) and pancreatic cancer (triangle) individuals using CPMG spectra 802 and ZGPR spectra 804. Because of the limited sample size of the benign lung disease and NSCLC samples, PCA and PLS were performed with spectra from normal, benign pancreatic disease, and PDAC groups. Integrated areas of metabolite resonances from the CPMG and ZGPR spectra were obtained from equal-sized binning analysis with the water and EDTA resonances excluded. PCA could not clearly separate into distinct differential clusters plasma metabolite data from normal, benign and pancreatic disease and PDAC individuals based on metabolic signatures.

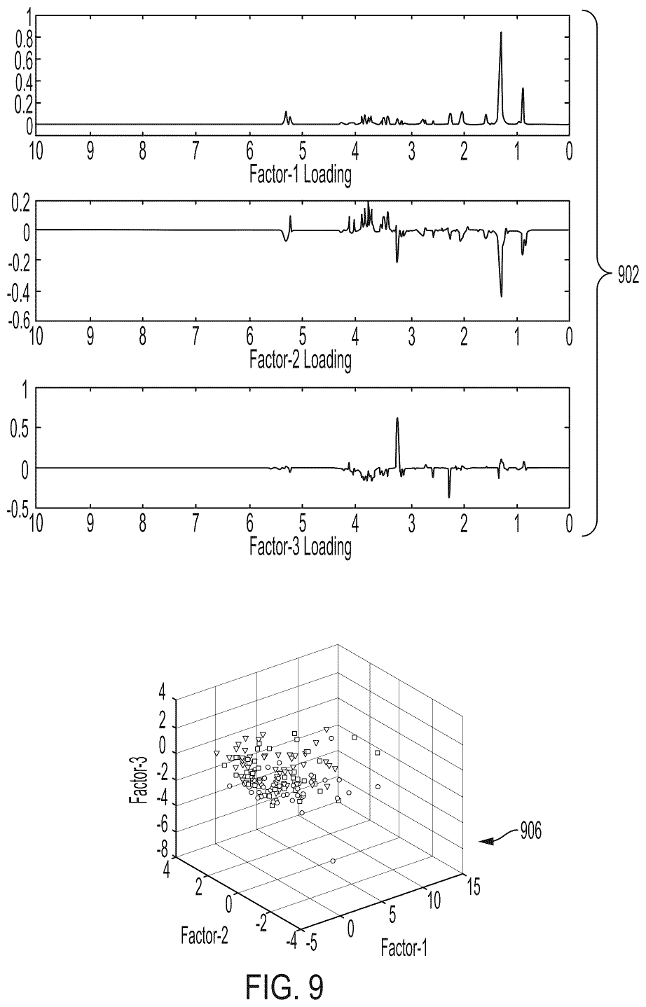

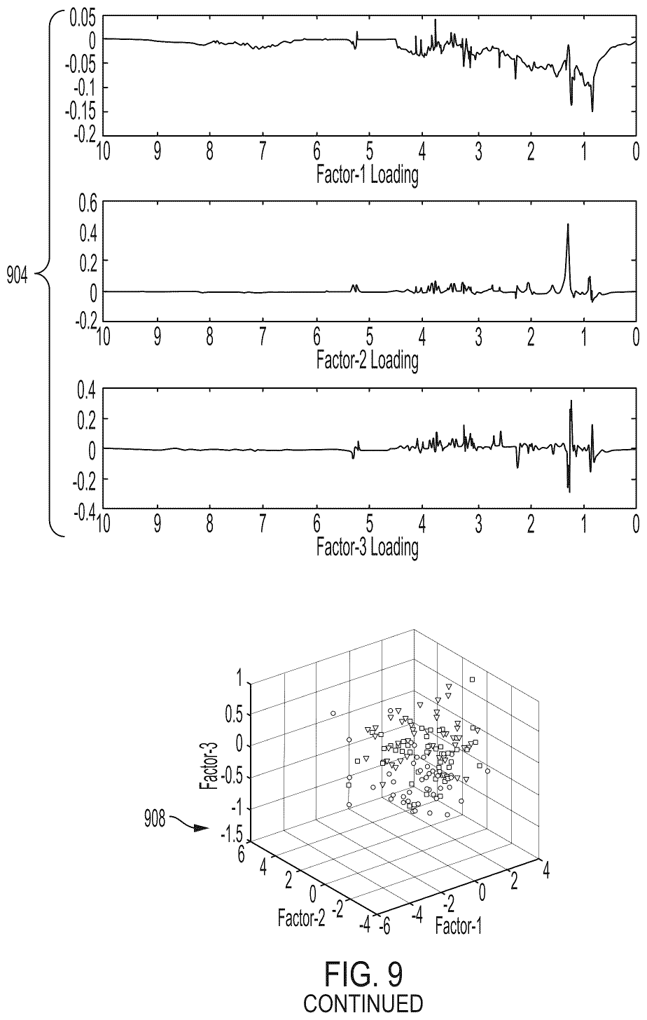

FIG. 9 depicts PLS loadings 902, 904 and scatter plots 906, 908 for supervised partial least squares regression analysis of normal, benign pancreatic disease, and PDAC NMR spectra. PLS loadings generated from PLS are shown for the spectral profiles acquired from CPMG spectra 902 and ZGPR spectra 904 from plasma of normal individuals, and individuals with benign pancreatic disease and PDAC. Scatter plots derived from PCA of spectra from plasma of normal (circle), pancreatic disease (square) and pancreatic cancer (triangle) are shown for individuals using CPMG spectra 906 and ZGPR spectra 908.

Supervised PLS analysis resulted in better clustering than PCA, although even supervised PLS was not able to distinctly separate the three groups. The loading plots allowed for identification of metabolites that contributed to differences between the groups. In general, these results demonstrate the high accuracy of the first reductions to practice in classifying the three groups compared to multivariate pattern recognition analysis of the spectra.

### III. Spectral Region Relevancy Analysis for the First Reductions to Practice

This section presents analysis of the spectral patterns associated with ppm regions that played discrimination-determining roles in determining the accuracy achieved by the neural networks of the first reductions to practice. In addition to providing a rational explanation for the neural network analysis, which supports clinical usage, identifying metabolites associated with these discrimination-determining spectral regions can expand the understanding of the systemic effects of cancer on metabolism.

To perform the analysis, in a first reduction to practice, all spectral regions that constituted the input feature vector were selectively suppressed one spectral region at a time. The resulting drop in neural network accuracy in the detection of PDAC and NSCLC was tabulated. Spectral regions that resulted in the largest accuracy drop were categorized as discrimination-determining spectral regions. Spectral regions were ranked from the highest to lowest in terms of decreasing accuracy, providing a set of spectral regions ranked according to their importance in deciding the accuracy of the neural network. Metabolites associated with these discrimination-determining spectral regions were identified. Achieving a zero-detection accuracy required suppressing all the spectral regions indicating that all spectral regions contributed to the classification of PDAC and NSCLC. Further, this section maps these regions to the corresponding loading plots obtained with PCA (see Section II).

FIG. 10 depicts discrimination-determining CPMG spectral regions 1000 identified by an analysis according to a first reduction to practice. In particular, FIG. 10 depicts representative CPMG spectral regions of interest 1002 identified to play a prominent role in classification accuracy. The bold lines represent the mean, and the dotted lines represent±1 SEM of each group, with thick curves representing malignant, medium curves representing benign, and thin curves representing normal spectra. FIG. 10 also depicts a mapping 1004 of discrimination-determining CPMG-obtained ppm spectral regions identified by a first reduction to practice to the corresponding loading plots obtained with PCA.

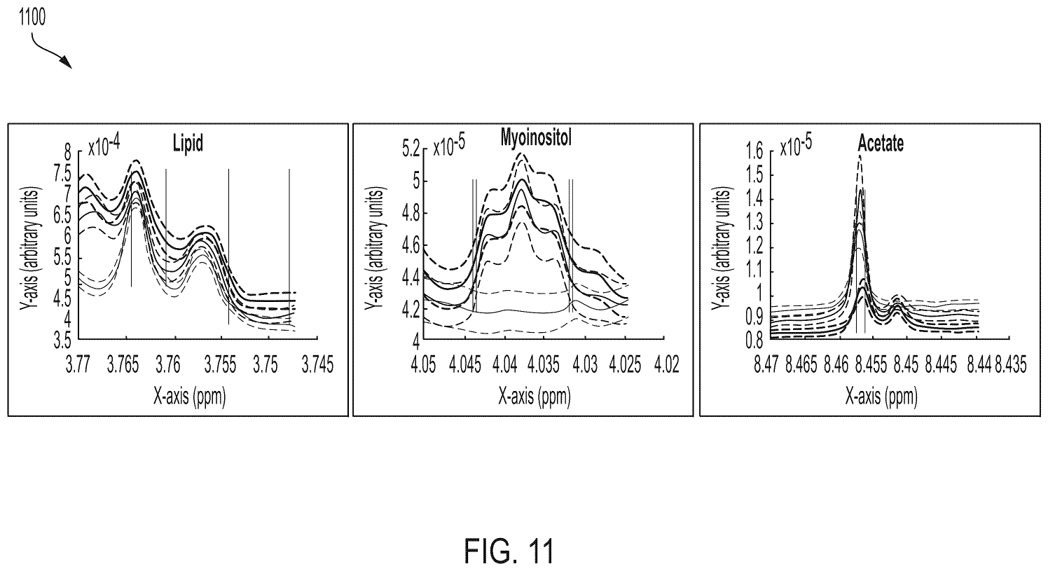

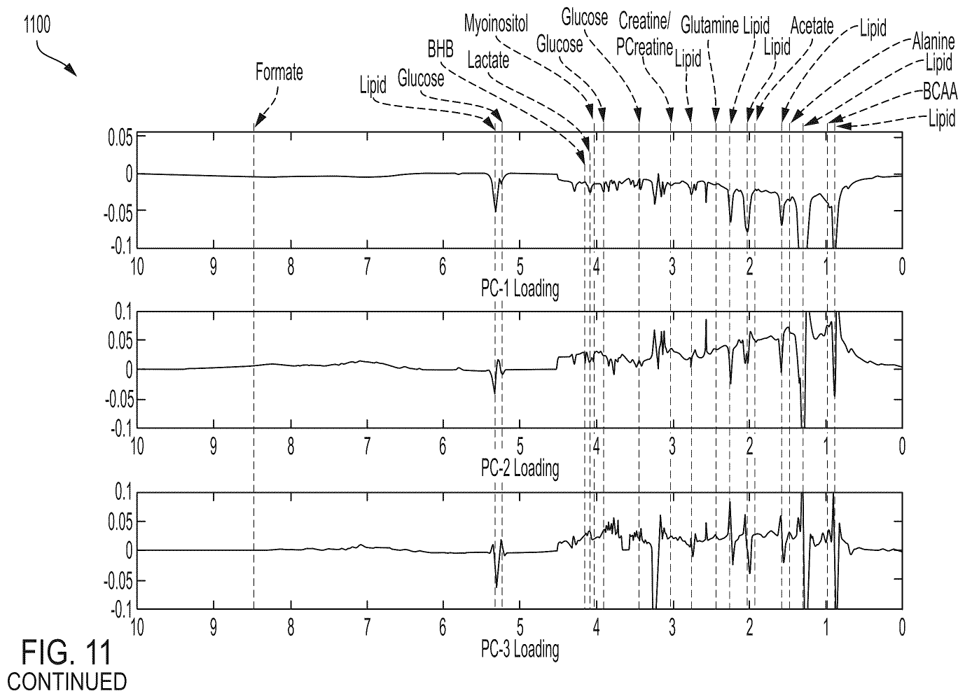

These results demonstrate that the first reductions to practice were able to separate the three groups based on spectral patterns identified in the lipids, glucose, lactate, acetate, citrate, pyruvate, creatine, glutamine, alanine, myoinositol, BHB (beta-hydroxybutyrate) and BCAA (brain chain amino acids) regions. Additionally, a spectral pattern difference in the glycine region was also identified from the CPMG spectra. Results from a similar analysis performed with ZGPR spectra are shown and described below in reference to FIG. 11.

The inventors performed a univariate analysis of the metabolites identified in the CPMG spectra, as shown in the table 700 of FIG. 7. Regarding the results of FIG. 10, the rightmost column of the table 700 identifies the association of metabolites that were identified as significantly different in the univariate analysis to discrimination-determining spectral regions identified by the first reductions to practice. Three amino acids with very low signal (tyrosine, histidine, and phenylalanine) were identified as significantly different in one or two of the comparison groups of the univariate analysis, but were not identified as being associated with discrimination-determining spectral regions in the analysis by the first reductions to practice.

FIG. 11 depicts discrimination-determining ZGPR spectral regions 1100 identified by an analysis according to a first reduction to practice. FIG. 11 depicts representative ZPGR spectral regions of interest 1102 identified by a first reduction to practice to play a prominent role in classification accuracy. As in FIG. 10, in FIG. 11, bold lines represent the mean and the dotted lines represent±1 SEM of each group, with thick curves representing malignant, medium curves representing benign, and thin curves representing normal spectra. FIG. 11 also depicts a mapping 1104 of discrimination-determining ZGPR-obtained ppm spectral regions identified by a first reduction to practice to the corresponding loading plots obtained with PCA.

The results depicted in FIG. 11 confirm the observations of FIG. 10, that spectral patterns identified in the lipids, glucose, lactate, formate, acetate, glutamine, myoinositol, BHB and BCAA regions played a prominent role in the accuracy of the first reductions to practice in detecting cancer. Additionally, a spectral pattern difference in the formate region was also identified from the ZGPR spectra.

### IV. First Reduction to Practice Neural Network Architecture

This section presents the architectures of example machine learning systems, which include neural networks, as used in the first reductions to practice. Before describing the neural network architecture of the first reductions to practice and their training, a description of acquiring and preparing the training, validation, and testing NMR spectral data is provided.

The first reductions to practice used human plasma and serum samples. Fasting plasma samples from individuals with no clinical evidence of pancreatic disease (normal, n=49), from individuals with benign pancreatic lesions (benign, n=49), and from individuals with PDAC (PDAC/malignant, n=53) and fasting serum samples from individuals with benign lung lesions (benign, n=11) and from individuals with NSCLC (n=22), were analyzed with ¹H NMR spectroscopy. Serum samples from the NSCLC individuals were obtained pre-operatively and with no exposure of individuals to chemotherapy. Plasma samples from 16 PDAC individuals were obtained prior to chemotherapy, with 37 serum samples obtained with at least a one-month interval after chemotherapy. ¹H MR spectra of plasma were acquired on a Bruker Avance III 750 MHZ (17.6 T) NMR spectrometer equipped with a 5 mm probe. Serum spectra were acquired on a Bruker 500 MHZ (11.7 T) NMR spectrometer equipped with a 5 mm probe. Plasma or serum samples (250 μL) were diluted with D20 buffer (350 μL) and spectra with water suppression were acquired using a ZGPR pre-saturation and a single pulse sequence with the following parameters: spectral width of 15495.86 Hz (8012 Hz for spectra acquired at 500 MHZ), data points of 64 K (32K for spectra acquired at 500 MHZ), 90° flip angle, relaxation delay of 10 s, acquisition time 2.11 s (2.0447 s for spectra acquired at 500 MHZ), 64 scans with 8 dummy scans, receiver gain 64 (80.6 for spectra acquired at 500 MHZ). Spectra were also acquired using a one-dimensional CPMG pulse sequence with water suppression with all other acquisition parameters as above. Spectral acquisition, processing and quantification were performed using TOPSPIN 3.5 software. Area under peaks were integrated and normalized with respect to the reference signal.

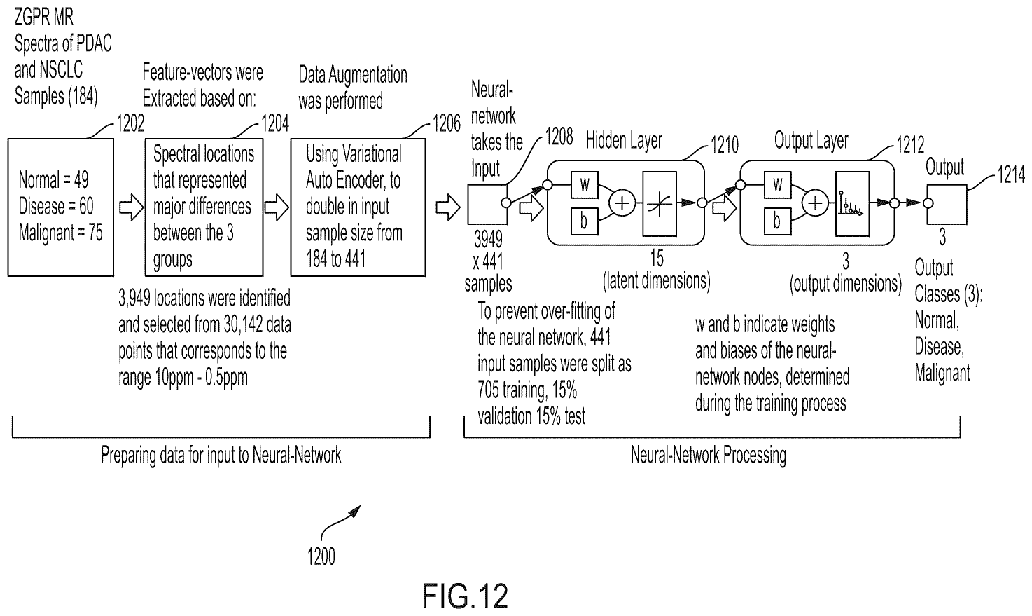

FIG. 12 is a schematic diagram of a machine learning system 1200, including a signal-channel artificial neural network, according to the first reductions to practice. The left half of the diagram illustrates data preparation, and the right half illustrates the neural network. These portions are described presently with respect to both application of the machine learning system 1200 to a novel input and to the training of the neural network. Note that blocks 1202 and 1206 are unique to training the neural network based on a set of training samples and may be omitted for application of the machine learning system 1200 to novel NMR spectra for assessment.

Application of the machine learning system 1200 to evaluate a novel NMR spectrum is described presently. After a patient's biofluid is extracted and an NMR spectrum is obtained, e.g., as described above in this section, the spectrum data is sampled, e.g., at regular intervals, from 10 ppm-0.5 ppm. In the first reductions to practice, 30,142 sample location data points were used. The sampled NMR spectrum data is then passed to a feature scaling block 1204.

The feature scaling block 1204 was applied to the spectral data as a pre-processing step, prior to the neural network analysis, to obtain a feature vector from the sampled NMR spectral data. For the parameters of the feature scaling block 1204 in the first reductions to practice, spectral data from each group (normal, benign disease, cancer) of the training spectra were centered around the mean with unit standard deviation. Mean spectra from each classification group were calculated, and differences between the means of the disease and malignant groups from the normal group were calculated to identify spectral segments that exhibited significant differences. A threshold value, computed based on mean and standard deviation, was used for this purpose of assessing significant differences. In the first reductions to practice, 3,949 locations were selected from the 30,142 sampled locations using this criteria.

In training, because the feature vector may be biased toward the relative distribution of variations within each class (normal, disease, and malignant) in the training datasets, to reduce this effect, the training dataset was randomly shuffled, leaving out a small fraction of the dataset in each shuffle to determine the most frequently occurring sections of the feature vectors. The resulting feature vector that included only the most frequently occurring sections of the original feature vector can effectively represent the real-world variations in each class and was used.

Once the feature vector was obtained from the feature scaling block 1204, for evaluation of the patient's biofluid NMR spectral data, the feature vector was passed to the neural network processing as shown on the right side of FIG. 12, which took the pre-processed input and creates a latent dimension representation before mapping it to the final output into three classes or groups. Thus, the neural network included an input block 1208, which accepted 3949-dimensional feature vectors derived from ZPGR NMR spectra data by the feature scaling block 1204, and passed it to a hidden layer 1210. The hidden layer 1210 mapped the input feature vector to the reduced hidden fifteen-dimensional representation, and the output layer 1212 mapped the hidden dimensions to a final three output dimension corresponding to normal, benign disease and malignant disease groups. The neural network thus provided an output 1214. In general, according to various embodiments, the output may be in the form of an electrical signal, a displayed indication, or any other form of electronic communication of the determined classification.

For training the neural networks of the first reductions to practice, spectral data were divided into three groups. For the CPMG spectra, the three groups were normal, benign pancreatic disease, and PDAC. For the ZGPR spectra, the three groups were normal, benign pancreatic and lung disease, and PDAC and NSCLC. NMR spectra from plasma or serum samples were normalized with respect to the reference signal and calibrated with respect to the sample volume. Additional verification was performed to ensure that the spectral data were represented as a linear array of data points with identical array size (30,142 elements) and ppm range (10.0 ppm-0.5 ppm) with equal-size binning. Identical dimensions and ppm ranges were maintained across all samples, and the number of samples per group was maintained approximately the same across all three groups. This was used to minimize any biases in the neural network during the training process.

The processing pipeline illustrated in FIG. 12 was used for combined PDAC and NSCLC ZGPR spectra. To train the neural network, the total input samples (the original and augmented data) were divided into proportions of 70%, 15%, and 15%, for training, validating, and testing the neural network. The neural network training process was repeated to achieve optimal fitting with maximum accuracy for the three classifier groups (normal, disease and malignant).

A similar neural network training approach was used for the PDAC CPMG spectra using corresponding input sample sizes. The main differences were the number of samples and the size (number of elements) of the feature vector. In the case of CPMG spectra, the feature vectors were doubled for each class (see description of variational auto encoder processing 1206 below) and any excess over the least three was ignored to create an equal number of 98 feature vectors per class.

For training, the processing started from the left of FIG. 12, with the source input data 1202 from which the feature-vectors were extracted by feature scaling block 1204. According to the first reductions to practice, variational autoencoder processing 1206 was used to create equal numbers of feature vectors for each class before feeding the feature vectors to the neural network. Further, to enhance the accuracy and robustness, and to minimize training biases in the neural network, variational auto encoder processing 1206 was used to approximately double the sample size of each group. The variational auto encoder processing 1206 provided a Gaussian distribution approach for describing the feature vectors in a latent space, such that new feature vectors were generated to probabilistically mimic the original feature vectors. This allowed the creation of augmented data that preserved the normal distribution of the groups in feature space. The number of additional feature vectors generated for each group during the variational auto encoder processing 1206 was such that the total number of samples were equal for the three groups. This effectively resulted in doubling of the input samples in the case of PDAC CPMG data analysis. In the case of combined PDAC and NSCLC ZGPR data analysis, either a doubling or tripling of input sample sizes was performed due to the appreciable differences between the original sample size from each class.

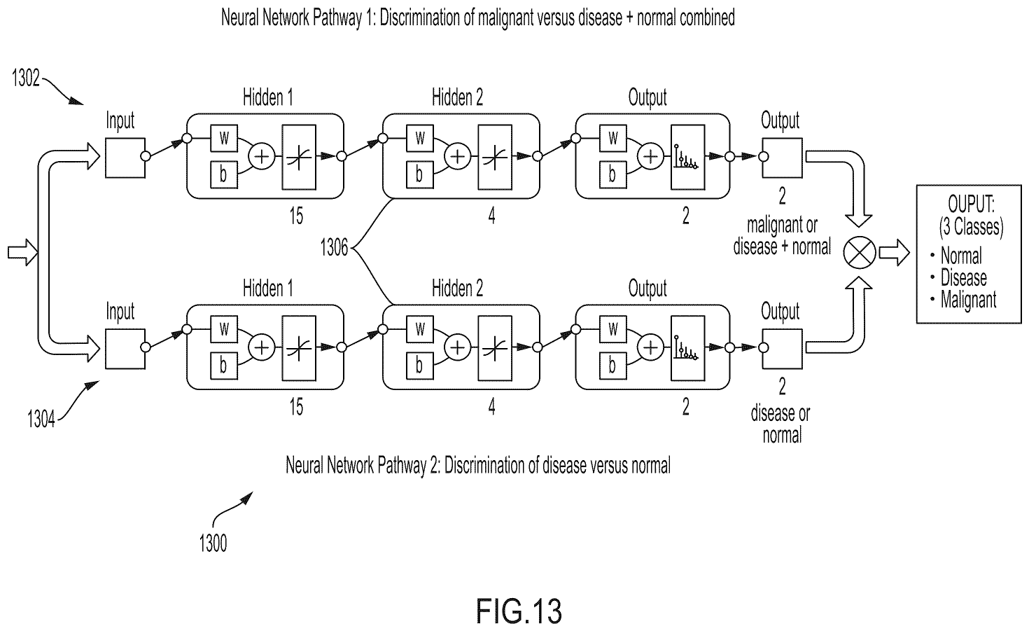

FIG. 13 is a schematic diagram of a machine learning system, including a two-channel artificial neural network, according to an example embodiment. In general, the artificial neural network as shown and described in reference to FIG. 12, can meet the requirements for classifying the training datasets and may be employed according to various embodiments. To further address variations that can arise in real-world test samples, a modified artificial neural network as shown in FIG. 13 is presented. The modified artificial neural network is particularly suited to solve two problems that can arise in real-world clinical data. The first problem is that disease class samples may sometimes fall in between normal and malignant class in the discrimination process, and these samples can be difficult to tease apart from the two other classes in a single neural network pipeline. To solve the first, a dual-network artificial neural network pathway 1300 is presented so that malignant class is discriminated against the other two classes separately in a first network path 1302 and the disease versus normal discrimination is processed separately in a second network path 1304. The second problem is that the variations in the samples within each class can grow larger as the sample sizes grow. To make the artificial neural network flexible to adapt to such variabilities within each class, a second hidden layer 1306 is introduced in the network pipeline. The second hidden layer 1306 translates the 15 dimension output from the first hidden layer into a 4 dimensional representation before classifying the samples in two groups in each respective network path 1302, 1304. The first network path 1302 discriminates malignant against the combined disease and normal class. The second network path 1304 discriminates the disease versus normal class. The final outputs from the two network paths 1302, 1304 are merged together to provide the intended three way classification: malignant, disease, or normal.

### V. Discussion Regarding the First Reductions to Practice

The first reductions to practice disclosed herein provided accurate discrimination of normal, benign and malignant classes with a sensitivity of 100%, 98.6%, 100% and specificity of 99.6%, 100%, 99.6% respectively. Further, a set of spectral regions in the source spectral data that played a major role in the discrimination was identified.

The first reductions to practice analyzed the ppm range of each NMR spectrum in substantially their entireties and at their full resolutions. The only omitted portions of the ranges were for the solvent (water) and a contaminant (EDTA). In general, embodiments may utilize the entire NMR spectral range, e.g., the entire NMR spectral range except for regions for a solute (by way of non-limiting example, water, methanol, or acetonitrile) and any contaminant(s) (by way of non-limiting example, EDTA). The approach of the first reductions to practice did not restrict the analysis to a select set of spectral ranges such as those corresponding to a preferred set of metabolites as probable candidates, nor reduce the resolution of the spectra. Both of these strategies are frequently employed to minimize the computational complexity of the analyses, however, both reduce accuracy. An advantage of the approach of the first reductions to practice is that it first trained a neural network to provide the highest possible accuracy and then used the trained neural network to identify parts of the spectra that played a prominent role in determining the accuracy of the neural network. This eliminated the need to fine-tune the neural network individually for multiple sets of possible spectral ranges to identify spectral ranges that potentially played a major role in determining the overall accuracy of the neural network.

Spectral patterns that played a role in discriminating between the three groups were associated with lipids, glucose, lactate, acetate, glutamine, myoinositol, BHB and BCAA regions in both ZGPR and CPMG spectral analysis. Glycine and citrate were the only metabolites identified in CPMG spectra that was not identified in the ZGPR spectra. Similarly, formate was the only metabolite identified in ZGPR spectra that was not identified in CPMG spectra. The commonality of these metabolites across both types of acquisitions provided further confidence of their contribution to the discrimination, providing additional validation of the analysis. While the PCA loading plots as well as univariate analysis of individual metabolites identified some of these differences, clear clustering of the three groups was not evident, unlike the analysis by the first reductions to practice, which identified the three groups with an average specificity of 99.7%.

The first reductions to practice both used substantially the entire spectral range to achieve accuracy and were used to identify discrimination-determining spectral regions that drove the accuracy. Both accuracy and the ability to explain the results are equally useful for gaining clinical acceptance. The results strongly support that first reductions to practice met the demands of accuracy required in the clinical use of a blood-based diagnostic technique to detect hypermetabolic cancer, such as PDAC and NSCLC.

### VI. Description and Architecture of the Second Reduction to Practice

The second reduction to practice described herein utilized a three-channel artificial neural network architecture. The second reduction to practice was trained to classify a blood plasma ZGPR NMR spectrum into one of the following three classes: normal, benign pancreatic disease, or PDAC.

The implementation of the second reduction to practice was similar to that of the first reductions to practice, with relevant distinctions described in this and the following sections. Among other differences, the neural network of the second reduction to practice included three channels, to accommodate the three-way classifications (normal, benign disease, and malignant). Further, the second reduction to practice utilized particular conditioning of the input data so that it met the profile characteristics of the original training data. Yet further, the second reduction to practice was validated using blinded test samples, which provided spectral data that the neural network had not encountered in its training phase.

For the second reduction to practice, a total of 170 human plasma samples were analyzed with proton (¹H) NMR spectra. The samples represented three groups of participants:
(i) participants with no clinical evidence of pancreatic disease (normal, n=58),
(ii) participants with benign pancreatic lesions (benign, n=53), and
(iii) participants with PDAC (PDAC/malignant, n=59).

All analyses were performed using de-identified human blood plasma samples.

While the second reduction to practice is described in reference to ZGPR spectral data, such description is non-limiting. The second reduction to practice may be applied to both CPMG and ZGPR NMR spectra acquisition methods.

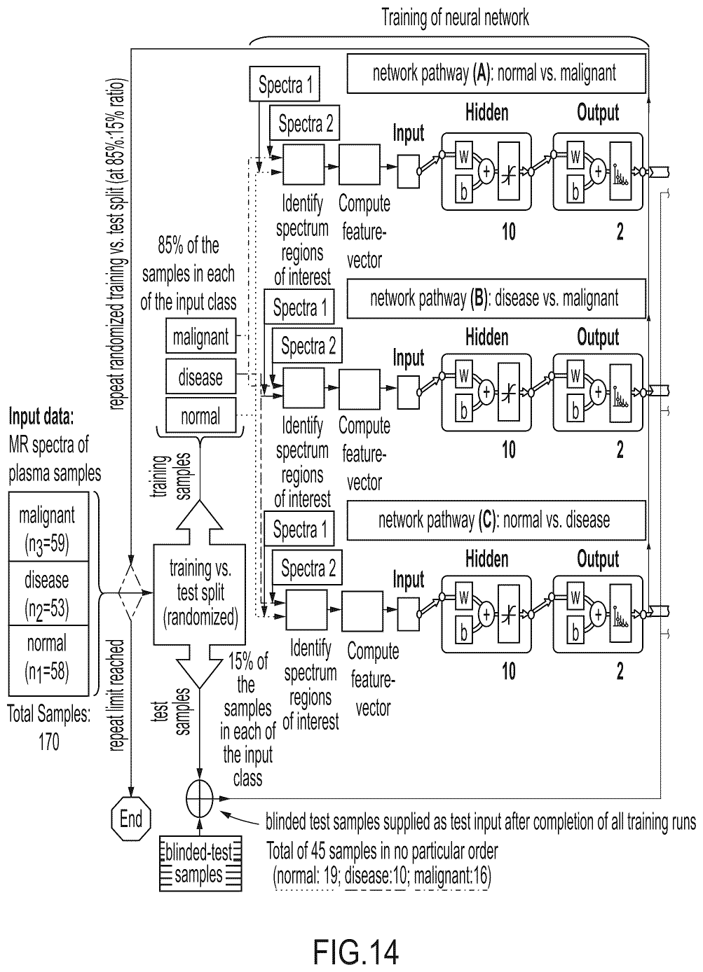

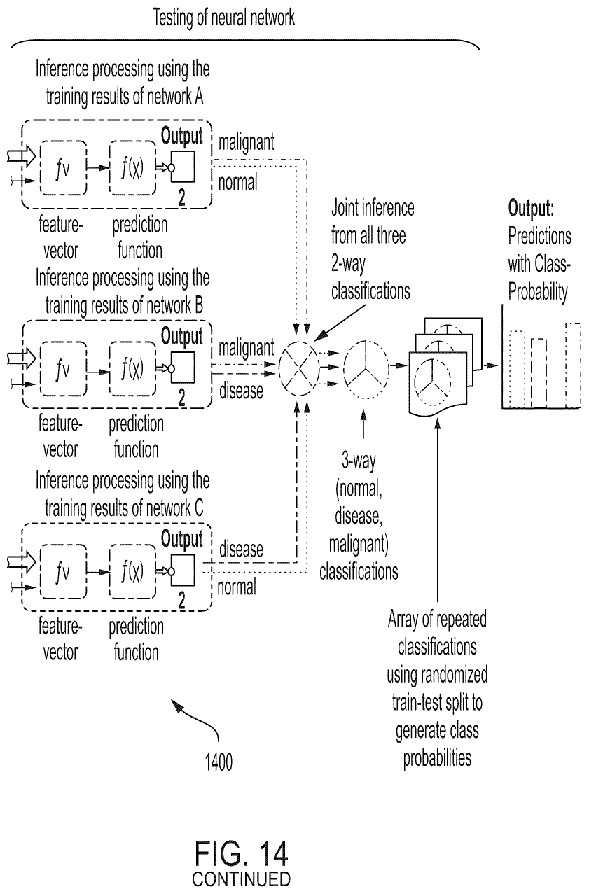

FIG. 14 is a schematic diagram of a machine learning system 1400, including a three-channel artificial neural network, according to the second reduction to practice. According to various embodiments, each channel (also referred to herein a “pathway”) may represent an independent artificial neural network. In the second reduction to practice, the three individual artificial neural network channels together provided a three-way discrimination of normal, disease and malignant. Configuring the neural network architecture with three neural network pathways allowed for fine-tuning each individual neural network channel to solve a two-way classification problem independent of the other two classification problems: normal or malignant (pathway A), disease or malignant (pathway B) and normal or disease (pathway C). The output from the three neural network pathways was combined together to arrive at the final output for the three-way discrimination result.

The spectral data used for both training and inference according to the second reduction to practice underwent data conditioning as follows. During post processing that follows the spectral acquisition process, certain regions of extremely high peaks in the spectra (such as water signals) are suppressed in the spectral data conversion step, since these regions are not of significant value in the intended classification analysis and their presence in the data can cause numerical accuracy arising from its very large dynamic range. But the data points in the immediate vicinity of the suppressed regions can slightly vary in newly acquired data compared to the training data and that can introduce an artificial bias in the data. To make sure that these regions do not play any such role, the locations of these specific spectral regions are tagged to appropriately exclude these regions in any part of the analysis. In the second reduction to practice, these regions include water and EDTA (a contaminant from blood collection tubes) related resonance regions in the spectra together with small surrounding intervals.

The spectral data used for both training and inference according to the second reduction to practice underwent data preprocessing as follows. Spectral data can sometimes have strong peak signals in one sample but not in other samples. It may be related to certain underlying features in the sample, or it may be an artifact arising from the sample preparation process that cannot be uniquely identified. An oddly occurring peak may or may not play a role in training or inference depending on where it occurs in the spectra. However, its presence in the spectral data may contribute to slight differences in the dynamic range and the baseline level of the spectra.

Thus, in general, since the composition of peaks in every sample's spectrum is different, the baseline level of the spectrum can have slight differences with respect to other spectra. To make sure that this does not affect the analysis, as a preprocessing step, the spectra data from all samples may be represented in a uniform Cartesian coordinate frame of reference. This helps ensure that the differences between two spectra can be accurately determined without bias arising from baseline differences. For this purpose, for the second reduction to practice, every spectrum was standardized using the mean and standard deviation of the spectrum. Mathematically, if S represents an array of data points from a spectrum, and if Sₘₑₐₙ and S_{std} are the mean and standard deviation of the data points of the spectrum S, then the standardized spectrum data may be computed as, by way of non-limiting example: (S−Sₘₑₐₙ)/S_{std}. These computations may be conducted pointwise, that is, the value of S may range over the amplitudes determined at each point in the spectrum.

To train each neural network pathway in the second reduction to practice, the input samples were split into training and test datasets at a split ratio of 85%:15%, and the training sets were supplied as input into the corresponding neural network pathway as shown in FIG. 14. In the second reduction to practice, each neural network pathway included a two-layer neural network with a hidden layer of size ten nodes and an output layer with two output nodes.

Each pathway was trained using the labeled training dataset for its respective two-way classification task. That is, each pathway was trained using spectra corresponding to its two respective classes. For both training and inference, the spectral data were converted into feature vectors, with each vector corresponding to a single sample spectrum.

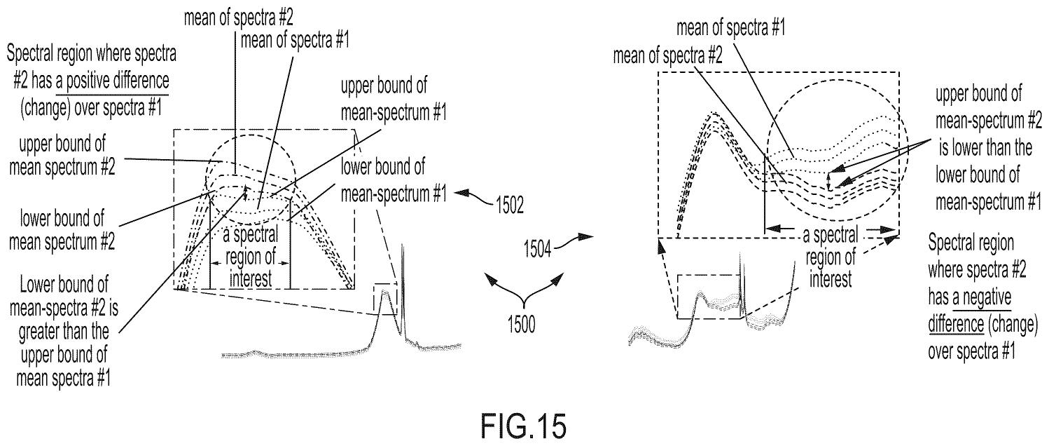

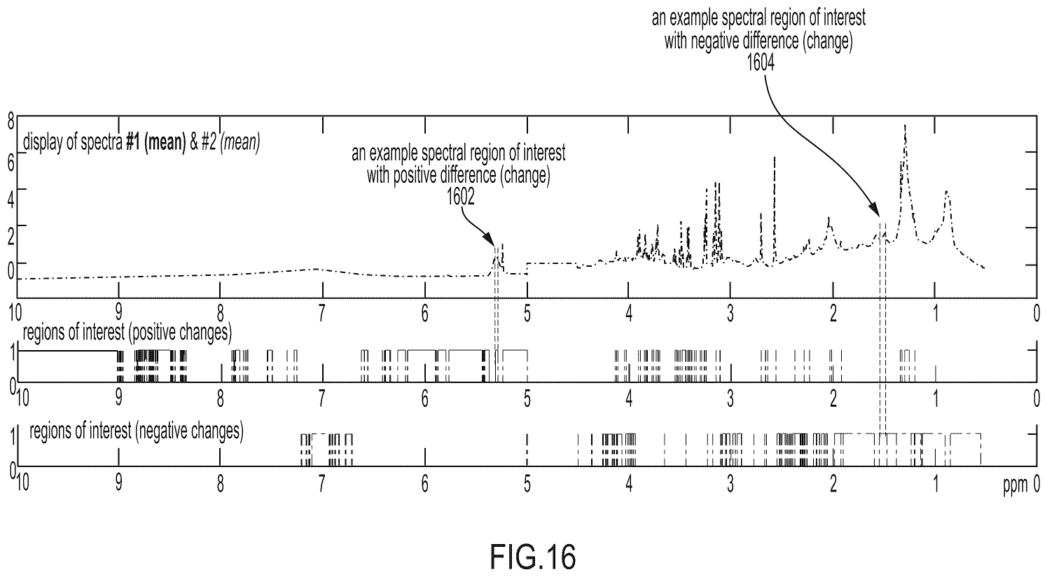

Training each pathway utilized a different version of the same process for generating feature vectors. More particularly, for each pathway, one class was regarded as the base class against which the other class, referred to as the comparison class, was compared, for purpose generating the feature vectors used for training. As shown in FIG. 14, and by way of non-limiting example, the bottom sub-path of each pathway represented the base class (denoted as Spectra 1, with the comparison class denoted as Spectra 2). Thus, for the top pathway (normal versus malignant) normal was considered the base class; for the middle pathway (disease versus malignant) disease was considered the base class; and for the bottom pathway (normal versus disease) normal was considered the base class. Each feature vector represented a collection of spectral differences between, on the one hand, the spectrum of the corresponding sample and, on the other hand, the mean of the spectra of the base class, computed at various spectral regions where the differences can be considered as significant, based on a well-defined criterion. Using the feature vectors, each pathway was trained to discriminate between the two classes to produce its corresponding classification. A detailed description of the process used to generate feature vectors as outlined above in reference to FIGS. 15 and 16 follows.

Feature vector generation for the second reduction to practice was performed individually for each channel. In general, the feature vector generation for a particular channel included two main steps. First, the spectral regions that showed sufficient differences between the spectra of the two classes for a channel were identified. Such regions are referred to herein as spectral regions of interest (SROI). Second, for a given individual spectrum for a sample, the corresponding feature vector was derived according to differences between the given individual spectrum and the mean of the base class spectra for the channel, computed for each of the spectral regions of interest. These steps are described in detail presently in reference to FIGS. 15 and 16.

FIG. 15 depicts a method 1500 of spectral region of interest determination according to the second reduction to practice. The spectral regions of interest with respect to two classes of a given channel were determined as follows (and the spectral regions of interest were so determined for each of the three channels and their respective classes). The pointwise mean and standard deviation of the training spectra over each individual class were computed. (Note that, in contrast to the mean and standard deviation computed for the preprocessing standardization described above, which are computed pointwise over a single spectrum, the mean and standard deviation here are computed pointwise over the entire set of spectra in the class.) The results of these computations were used to provide three spectral curves for the training spectra of each class: a mean spectral curve, a lower bound spectral curve, and an upper bound spectral curve. The upper and lower bound spectral curves were computed as the mean spectral curve plus and minus (+/−), respectively, the standard error (SE, standard deviation divided by number of samples) of the training spectra and multiplied by a scale factor. The scale factor was determined (using a range between one and the number of samples) based on the most optimal results during training. The upper bound spectral curve for the base class and lower bound spectral curve for the comparison class were compared against each other for mutual intersections (e.g., crossings) 1502, 1504 (in FIG. 15, #1 denotes base class and #2 denotes the comparison class). Wherever the lower bound of the comparison spectral curve was above the upper bound of the base spectral curve between two such intersections, the corresponding region was marked as a spectral region of interest with positive differences (positive spectral region of interest), e.g., 1502. Similarly, regions between two such intersections where the upper bound of the comparison spectral curve was below than the lower bound of the base spectral curve were marked as a spectral region of interest with negative differences (negative spectral region of interest), e.g., 1504.

FIG. 16 depicts positive and negative spectral region of interest groupings as used in the second reduction to practice. The contiguous positive spectral regions of interest were grouped together (e.g., 1602), and the contiguous negative spectral regions of interest were grouped together (e.g., 1604). Thus, the contiguous regions of the same type of difference (positive or negative) were grouped together to represent each as a respective contiguous spectral region of interest. The collection of such contiguous spectral regions of interest taken from over the entire range of the spectrum represented all regions where the two spectra may be considered to significantly differ from each other. For the second reduction to practice, and by way of non-limiting example, the number of spectral regions of interest was between 100 and 600 for various combinations of training samples, with a more common range between 200 and 300. Thus, the first step for determining feature vectors—identifying the spectral regions of interest—was performed as described for the second reduction to practice. In general, this step may be performed once, during the training phase, and the second step, deriving a feature vector for a given spectrum of a sample, may utilize the same identified spectral regions of interest from the first step, both during the training and the inference phases, to compute corresponding feature vectors.

The second step, deriving a feature vector for a given spectrum of a sample, is described presently. For the second reduction to practice, for a given spectrum of a sample, the corresponding feature vector was derived as a vector representing the sum, over spectral locations, of the differences in the amplitudes between the given spectrum and the mean of the spectra of the base class, computed for each of the spectral regions of interest. Therefore, the number of elements in the feature vector was equal to the number of spectral regions of interest. Because an individual spectral region of interest defines a contiguous set of spectral locations, the summing up operation on the differences in the amplitudes can be regarded as computing the difference in area between the given spectrum and the mean of the base class spectra, within the spectral region of interest.

Thus, for the second reduction to practice, a feature vector for a given spectrum represented the differences in area between the curve of the given spectrum and the mean curve of the spectra of the base class, evaluated at each of the spectral regions of interest. In the second reduction to practice, a summation of the amplitudes in the consecutive spectral locations of a contiguous spectral region of interest corresponded to the area under the curve. This is due to the high resolution nature of the NMR spectra used in the analysis. In general, if the spectra used in the analysis happens to be of lower resolution, or if a more accurate assessment of the area under the curve is desired, for example, an analytical method for computing the area, (such as trapezoidal rule and/or spline fitting of spectra) may be implemented to derive an improved form of feature vector.

A feature vector, computed as explained above, may be supplied as input to the neural network as shown in FIG. 14 for training or inference. The feature vector, in its data representation, maintains two separate groups within the vector array, one for the positive differences and the other for the negative differences (both differences are illustrated in FIG. 16). In the second reduction to practice, the neural network processed the feature-vector without any explicit distinction within the processing pipeline between the positive and negative differences. In general, if a particular spectra classification application requires processing the two regions differently (e.g., to process either one of them but not both together), a provision made available in the data representation of the feature-vector may be utilized to allow for that flexibility in processing.

For the second reduction to practice, and in general, the spectral regions of interest and the mean of the base spectra may be considered as training state variables that may be used during the inference phase when predicting the classification of a new sample. Therefore, these parameters were stored in electronic persistent memory as part of the training process.

A detailed description of training the neural network of the second reduction follows. The randomized train/test split process was repeated (or iterated) several times so that any incidental convergence of the neural network under certain combinations of train/test samples got sorted out against the more stable convergence of the neural network on a broader range of train/test sample combinations. In general, although there is no upper limit on how many times this process may be repeated for an embodiment, to ensure the stability of the network convergence, it is noted when the number of repetitions exceeds the total number of samples (in this case, 170), the performance improvements may not be significant. In the second reduction to practice, separate training runs were performed with following number of iterations: 60, 170 and 850 to cover a broad range training runs, to study the effect or their contributions to the stability of the classifications.

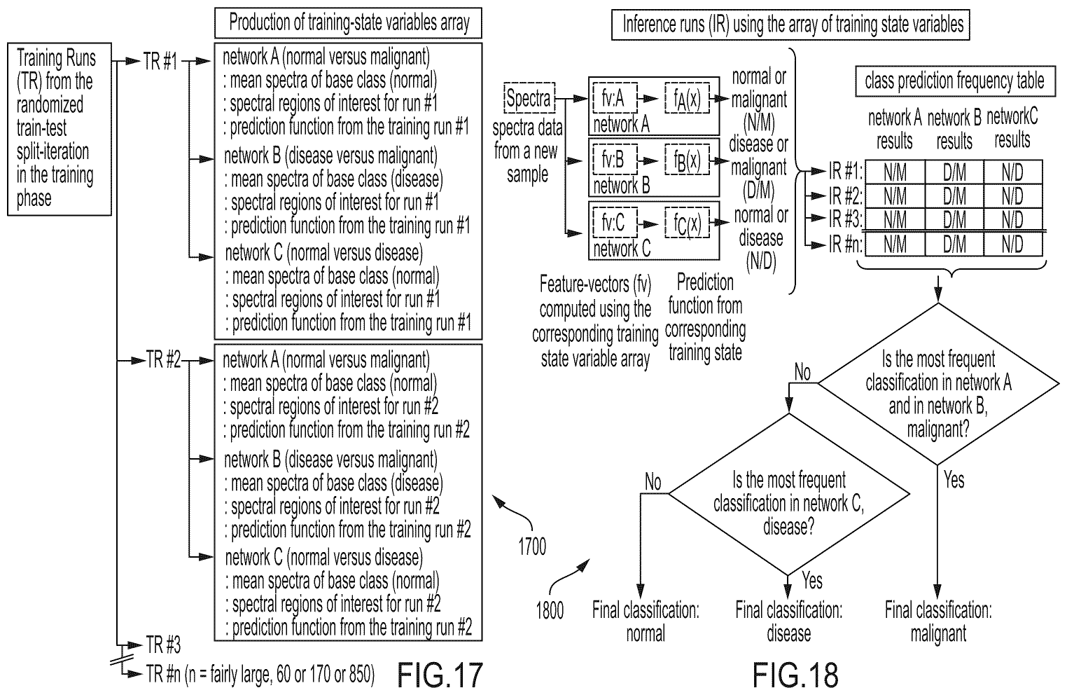

FIG. 17 schematically illustrates a method 1700 of neural network training as used for the second reduction to practice. The training runs produced training state variables, which are used in the inference phase when evaluating the classification of a new sample. From each training run, which uses a randomized set of train and test samples, the locations of the spectral regions of interest, the mean of the base class spectra, and the prediction function of the fully trained network (from the corresponding training run) were saved in the training state variable array in persistent memory.

For the inference phase according to the second reduction to practice, the spectrum of the sample to be classified was curated to properly align it with the spectra used for training. During the blinded test sample validation described herein in Section VIII, the inventors noted that small differences in alignment of the high-resolution NMR spectra could result in incorrect classification results. Therefore, for inference, the spectrum of the sample under investigation was curated to ensure that it maintained accurate spectral alignment with that of the original training dataset. In general, this curation may also be performed with the training spectra. To perform the alignment, selected reference metabolites, such as acetate or alanine, were used to ensure that the spectral peaks of the metabolites are perfectly aligned with those of the spectra samples used in the training.

A detailed description of the inference phase of the second reduction to practice follows.

FIG. 18 schematically illustrates the method 1800 of inference for classifying a new sample spectrum as used for the second reduction to practice. Using the training state variable array, the classification of a new or different sample (e.g., other than the ones used in the training) was determined as shown in FIG. 18. Depending on the combination of the train/test split, a test sample may get classified correctly (or incorrectly) depending on its similarity (or uniqueness) in comparison to the samples in the training set. Thus, for purpose of inference, the iterations over several times provided a reasonably reproducible classification for the test samples. The two-way classification result of a test sample in each of the neural network paths was determined based on its frequency of occurrence within the inference runs. That is, for classification of a sample, the inference runs were performed multiple times, once for each corresponding training iteration, using the associated training state variables, to compute the feature vector and input it to the corresponding prediction function (that was obtained from trained neural network) to determine its classification in each inference run. The most frequently occurring classification from the multiplicity of inference runs determined the results of the two-way classification.

The final three-way classification of a test sample according to the second reduction to practice was determined as follows. If a sample got classified as malignant in both network-A (normal versus malignant) and in network-B (disease versus malignant), then that sample was assigned to malignant class. Otherwise, its classification was determined based on the classification of network-C (normal versus disease) to assign a final classification of normal or disease. The final classification was indicated by display on a computer monitor. In general, according to various embodiments, the indication may be in the form of an electrical signal, a display, or any other form of electronic communication of the determined classification.

An advantage of the repeated training runs with different (randomized) train/test split combinations in the second reduction to practice was that, in addition to determining highly reproducible classification results for a test sample during the inference phase, a probability number could be assigned to the classification of the test sample based on its frequency of occurrence in different channels of classifications. Thus, for example, if a sample got classified as either normal or disease, 50% of the time each, it may represent that either the neural network was unable to converge with higher confidence (based on the current training dataset) or that the sample represented a borderline condition. Thus, class probability represents useful secondary information in conjunction with the primary classification. For example, with the appropriate large training set of spectra, the class probability may accurately represent a confidence in the classification. According to some embodiments, the class probability may be output for consideration by a clinician or other individual to whom it may be of concern.

### VII. Accuracy Boosting of the Second Reduction to Practice

During the iterative training runs, with random train/test split samples, the inventors observed that some of the samples frequently (e.g., 100% of the time) got misclassified when included in the test group. This was because these samples represented distinct characteristic features of the corresponding class that were not commonly occurring in other samples in the class. As a result, when such a sample was not included in the training set, it did not get correctly classified. The spectral features of these samples may represent distinct and less-common variants of the spectral features of the class. The number of such samples per class was small compared to the total number samples (e.g., 25%). By always including such samples in the training set, the accuracy of classification of the other samples also improved. This was because, often, a few test samples might get incorrectly classified by a narrow margin in their frequency of occurrence during the inference runs. In these cases, the spectra of those training samples with less-common features improved the accuracy of classification sufficiently to push the frequency of occurrence in the inference runs above the needed threshold to result in the correct classification of the test sample. Thus, the combined net effect contributed to an overall accuracy boost. Such samples are referred to as pivot samples, because their presence or absence in the training set can result in a significant difference in the final accuracy of the trained neural network.

In general, pivot samples may be identified through a trial run of the training phase, with repeated iteration of the randomized train/test split. Those samples that consistently (or most frequently) fail in classification whenever they are included in the test samples may be identified as pivot samples. Pivot samples may be different for each neural network pathway. For instance, the pivot sample set for normal versus malignant and disease versus malignant classifications may be different from the pivot samples for normal versus disease. Thus, the classification performance can be significantly improved by using one set of pivot samples to determine whether a sample is malignant, and if it is not then switch to using another set of pivot samples to determine if that sample is normal or disease.

### VIII. Validation of the Second Reduction to Practice

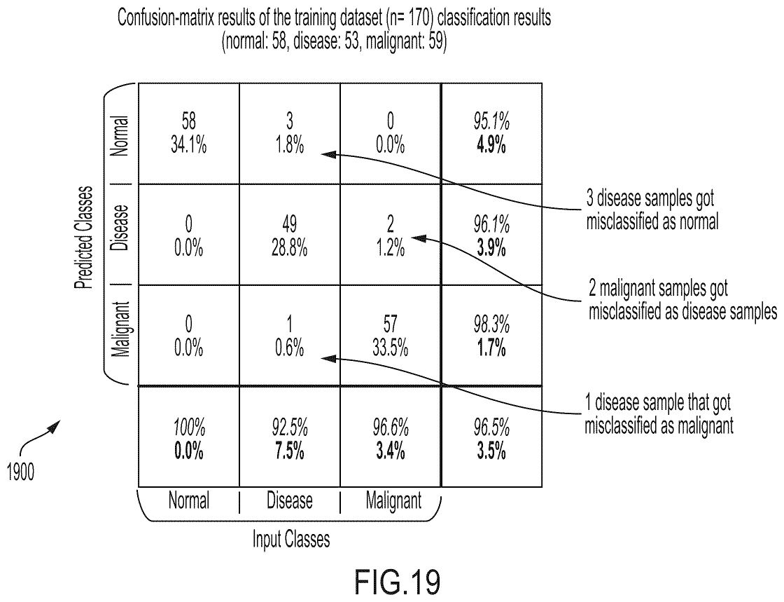

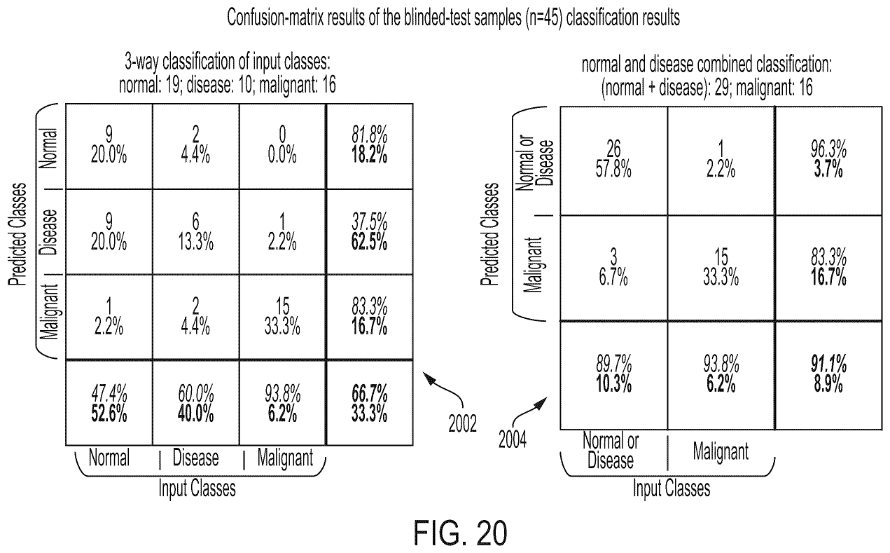

As set forth in this section, the second reduction to practice was validated in two ways. First, the second reduction to practice was validated based on the test samples of the train/test splits implemented during the training phase. The results of this validation are shown and described in reference to FIG. 19. Second, the second reduction to practice was validated using blinded test samples, which were not used at all during the training phase. The results of this validation are shown and described in reference to FIG. 20.

The validation results from the training phase and using the blinded test samples are demonstrated in the form of confusion matrices as shown in FIGS. 19 and 20, respectively. In general, each confusion matrix illustrates how the input samples (listed along the horizontal axis) are classified by the neural network (listed along the vertical axis). In each matrix, the three squares along the diagonal from the upper left to the lower right represent correct classifications, and the remaining squares represent misclassifications.

FIG. 19 shows a confusion matrix 1900 corresponding to the training phase validation of the second reduction to practice. Using the training state variable array, shown in FIG. 17, the classification of the test portion of the train/test sample splits were performed as shown in FIG. 18. The inference runs performed on the test data portion of the train/test splits acted as complement parts to the training runs performed on the training data portion of the train/test splits. The training state variable array that was produced during the training runs was applied on to a test sample in the corresponding inference runs.

During the inference runs, using the saved the data in the training state variable array, feature vectors were determined for the test spectra. The feature vectors were then processed through the saved prediction function for the corresponding neural network pathway (network-A, network-B or network-C). This provided the results for all three channels, and the results were entered in to a class prediction frequency table, as illustrated in FIG. 18. The inference runs were repeated through all of the training runs in the training state variable array. The final two-way classification results for each channel were determined based on the frequency of occurrence of the classification in the respective inference runs for the respective neural network pathway. The final three-way classification result was obtained as shown and described in reference to FIG. 18.

The results of this test sample classification during the training phase are illustrated in the confusion matrix 1900 of FIG. 19. In the training phase, the test samples were from the randomized train/test splits of the full training set of 170 samples, with 58 normal samples, 53 disease samples, and 59 malignant samples. The splitting ratio was 85%:15% for each class. In each training run, the training subset was used for the training and the test subset was used exclusively for testing. Thus, the test subset samples did not participate in the training of the network; therefore, these samples may be considered as unseen by the neural network when interpreting the results. As the training runs were repeated several times, iterating through the randomized splitting of train/test samples, several of the samples in the full training set of 170 samples may have eventually played the role of qualified test samples (i.e., as samples that were not included in the training set in the corresponding training iteration). Thus, the classification results on them may be used to demonstrate the performance of the neural network of the second reduction to practice.

The confusion matrix 1900 illustrates that the neural network machine learning system of the second reduction to practice resulted in a final overall performance of 96.5% accuracy, with very few misclassified samples. These results include implementation of pivot-sample based accuracy boosting, in which a small number of samples in each class were always included in the training.

FIG. 20 shows confusion matrices 2002, 2004 corresponding to an inference phase validation of the second reduction to practice using blinded test samples. The blinded test sample validation utilized 45 test samples that were not previously exposed to the neural network. As additional considerations for a blinded-test, the spectra for these samples were acquired at different times, all later to the training samples, and the original clinical classification of the samples were kept confidential until after the testing results were obtained from the described embodiment.

Confusion matrix 2002 shows the three-way classification result. Several of the normal samples got misclassified as disease, while the accuracy of prediction on the malignant samples was in the greater-than-90% range (at 93.8%). The reason for the normal versus disease misclassification was found to be that the spectra data themselves showed changes in these samples that were quite similar to those of disease samples. One possible explanation is that these samples represented sub-clinical disease stages, and were therefore clinically considered as normal, whereas in the NMR spectra, they may appear as disease-related. Also, some of these samples included conditions such as diabetic or smoking-related lung diseases. The combined effect of these two conditions may have resulted in the misclassification of these samples.

It should be noted that when the normal and disease classes were combined into a single class (for instance, when prioritizing on the primary result with respect to malignant class identification) the accuracy of the neural network increased significantly. This is illustrated in the confusion matrix 2004. The overall accuracy in this classification was above 90% (at 91.1%) with just a few misclassifications.

The tables below present the class probabilities for each of the blinded test samples. The tables are grouped according to the primary classification (normal, Table 1; disease, Table 2, and malignant, Table 3). The most significant is the malignant classifications (Table 3). It can be seen in Table 3 that the malignant probabilities for some of the malignant samples are as high as 100%, and in others of the malignant samples, the malignant probabilities are closer to 50%, with the remaining part of their class probabilities mostly falling in to the disease class, suggesting that there may be considerable overlap in the spectral characteristics between disease and malignant classes that may result in this division of the class-probabilities between the two classes.

TABLE 1
Primary Classification: Normal

| Normal | Disease | Malignant |
|---|---|---|
| 99.41% | 0.59% | 0.00% |
| 87.65% | 12.35% | 0.00% |
| 78.24% | 21.76% | 0.00% |
| 84.71% | 15.29% | 0.00% |
| 85.06% | 4.75% | 10.19% |
| 95.28% | 4.71% | 0.01% |
| 62.94% | 37.06% | 0.00% |
| 74.12% | 25.88% | 0.00% |
| 100.00% | 0.00% | 0.00% |
| 75.00% | 0.00% | 25.00% |
| 73.53% | 26.47% | 0.00% |

TABLE 2
Primary Classification: Disease (Benign)

| Normal | Disease | Malignant |
|---|---|---|
| 3.64% | 84.74% | 11.62% |
| 0.00% | 99.41% | 0.59% |
| 0.59% | 99.23% | 0.18% |
| 0.00% | 98.24% | 1.76% |
| 28.82% | 71.18% | 0.00% |
| 0.49% | 81.86% | 17.65% |
| 0.00% | 74.71% | 25.29% |
| 0.00% | 98.24% | 1.76% |
| 0.00% | 98.24% | 1.76% |
| 25.62% | 73.39% | 0.99% |
| 1.75% | 97.41% | 0.84% |
| 3.06% | 83.50% | 13.44% |
| 15.60% | 79.14% | 5.26% |
| 20.42% | 76.00% | 3.58% |
| 24.71% | 75.29% | 0.00% |
| 3.80% | 88.55% | 7.65% |

TABLE 3
Primary Classification: Malignant

| Normal | Disease | Malignant |
|---|---|---|
| 0.00% | 3.53% | 96.47% |
| 17.98% | 7.93% | 74.09% |
| 18.49% | 9.58% | 71.93% |
| 0.25% | 20.93% | 78.82% |
| 0.00% | 0.00% | 100.00% |
| 15.65% | 18.47% | 65.88% |
| 0.03% | 1.73% | 98.24% |
| 0.00% | 40.00% | 60.00% |
| 0.00% | 0.00% | 100.00% |
| 0.00% | 40.00% | 60.00% |
| 0.89% | 1.45% | 97.66% |
| 0.00% | 0.00% | 100.00% |
| 0.00% | 0.00% | 100.00% |
| 0.84% | 47.12% | 52.04% |
| 3.56% | 18.08% | 78.36% |
| 0.00% | 0.00% | 100.00% |
| 0.00% | 3.53% | 96.47% |
| 0.00% | 10.00% | 90.00% |

### IX. Variations of the Second Reduction to Practice

Many variations of the second reduction to practice are possible. By way of non-limiting examples, this section lists some such variations, which may be applied to any embodiment in general, and the second reduction to practice in particular.

Several of the parameters that are used with the artificial neural network of the second reduction to practice can be manually specified, or determined and/or optimized through trial runs. Some such parameters may depend on the total samples size and the characteristics of the composition of the individual sample groups used for training. As a result, when the number of samples sizes increases, some parameters may be fine-tuned to improve performance for a particular collection of training groups. A list of non-limiting example parameters is included below to indicate that such parameters may be subject to change and/or fine-tuning.
(1) In the second reduction to practice, each spectrum of the training spectra data was standardized using its mean and standard deviation. This helped ensure that all spectra data were represented in a uniform coordinate reference frame for computing the differences between the spectra. This scheme can be removed or replaced with a different scheme, e.g., for a different set of training samples. In general, the scheme may be replaced by using another metric, e.g., to represent all spectra in a common coordinate reference frame.
(2) For computing feature vectors, the second reduction to practice used a summation of individual spectral amplitude differences to evaluate the difference in areas between spectral curves. Such an equivalence, between sums of differences and areas, is possible with high resolution spectral data such as those used in the current analysis. If the spectral resolution changes, or if a more accurate evaluation of the differences between the spectra is desired, suitable analytical approaches to computing the area under between curves can be implemented.
(3) Certain neural network parameters for the second reduction to practice, such as number of layers (for the second reduction to practice, two), the size of the hidden layer (for the second reduction to practice, ten output nodes) and for the training-test split ratio (for the second reduction to practice, 85%:15%) were determined based on trial runs to determine a suitable neural network setup for sufficiently accurate results. Any of these parameters can change and a new set of parameters can be determined, e.g., if the sample sizes increase.
(4) In the second reduction to practice, the results from the three separate neural network pathways were combined in to one three-way classification, where priority was given to identifying the malignant classification first, followed by the disease or normal classifications. This logical order was determined based on the results for the size and distribution of the training samples used in the second reduction to practice. If the distribution characteristics of the training samples change, then a different way of combining the results from the three neural networks may be used, e.g., to increase performance.
(5) In the training runs used for the second reduction to practice, the number of times the randomized train/test split was repeated ranged from 60 (about the average size of one class) to 170 (the total sample size) up to 850 (five times the total sample size). Although higher numbers may lead to higher accuracy, the computation time is a bottleneck. With the use of more powerful computers, these repetition (or iteration) rates can be increased to further improve the classification accuracy.
(6) When determining the classification of a spectra based on the inference runs (as shown in FIG. 18), the second reduction to practice used a simple majority in the class prediction frequency table to assign the classification for that sample for each channel. Although, for the second reduction to practice, the performance metrics of the neural network were not taken in consideration, they can vary appreciably between different training runs. Therefore, when filtering the classification results based on the frequencies of occurrence, the performance metrics of the corresponding network can also be factored in as criteria to select subsets of inference runs. Such a scheme may be implemented, e.g., if it increases classification accuracy.

In general, the above list is exemplary rather than exclusive, and may apply to embodiments in general, including, but not limited to, the second reduction to practice.

### X. General Variations

This section presents non-limiting example variations of embodiments disclosed herein, not limited to the first and second reductions to practice.

In general, embodiments may utilize one or more neural networks that include one, two, three, or more channels.

In general, the machine learning systems used by various embodiments are not limited to neural networks, nor are neural network embodiments limited to using neural networks configured or parameterized as disclosed herein.

In general, any type of nuclear magnetic resonance spectroscopy may be used according to various embodiments, not limited to ¹H nucleus. By way of non-limiting example, embodiments may utilize other nuclei, such as ¹³C (Carbon), ¹⁹F (Fluorine), or ³¹P (Phosphorus-31)) based nuclear magnetic resonance spectroscopy.

Although some of the embodiments disclosed herein use ZGPR spectra, embodiments are not so limited. For example, any form of water suppression using pre-saturation pulses, by way of non-limiting example, ZGPR or ZGCPPR, may be used, or may be omitted altogether, according to various embodiments.

Although some embodiments disclosed herein use CPMG spectra, embodiments are not so limited. For example, any form of translational diffusion suppression may be used, or may be omitted altogether, according to various embodiments.

Although some embodiments disclosed herein use blood plasma and blood serum, embodiments are not so limited. Any biofluid may be used, by way of non-limiting example, blood plasma, blood serum, urine, saliva, or milk, may be used, according to various embodiments.

Although some embodiments disclosed herein detect PDAC and NSCLC, embodiments are not so limited. Any hypermetabolic cancer may be detected, by way of non-limiting example, PDAC, NSCLC, or renal cancer, according to various embodiments.

According to various embodiments, detection of cancer may automatically trigger additional actions, such as clinical follow-up. Such clinical follow-up may include, e.g., requesting, scheduling, or obtaining a biopsy or radiological scan, such as a CT scan.

Certain embodiments can be performed using a computer program or set of programs executed by an electronic processor. The electronic processor may include, but not limited to, multi-processor and multi core configurations of CPUs (Central Processing Units) and GPUs (Graphics Processing Units) or a combination of both. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by an electronic processor.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In various embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including a higher level programming language such as MATLAB, an object oriented programming language such as Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112 (f). However, for any claims containing elements designated in any other manner, it is intended that such elements are not to be interpreted under 35 U.S.C. § 112 (f).

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A computer implemented machine learning method of detecting a hypermetabolic cancer based on a nuclear magnetic resonance spectrum of a patient biofluid, the method comprising:
obtaining a nuclear magnetic resonance spectrum of a patient biofluid;
providing the nuclear magnetic resonance spectrum to a machine learning system trained with a training corpus, the training corpus comprising a group of normal biofluid nuclear magnetic resonance spectra and a group of hypermetabolic cancer biofluid nuclear magnetic resonance spectra; and
supplying an indication based on an output of the machine learning system, wherein the indication is representative of whether the nuclear magnetic resonance spectrum of the patient biofluid is indicative of cancer.

2. The method of claim 1, further comprising providing clinical follow-up for the patient upon an indication of cancer.

3. The method of claim 1,
wherein the group of hypermetabolic cancer biofluid nuclear magnetic resonance spectra comprises pancreatic ductal adenocarcinoma biofluid nuclear magnetic resonance spectra, and
wherein the indication is representative of whether the nuclear magnetic resonance spectrum of the patient biofluid is indicative of pancreatic ductal adenocarcinoma.

4. The method of claim 1,
wherein the group of hypermetabolic cancer biofluid nuclear magnetic resonance spectra comprises non-small cell lung cancer biofluid nuclear magnetic resonance spectra, and
wherein the indication is representative of whether the nuclear magnetic resonance spectrum of the patient biofluid is indicative of non-small cell lung cancer.

5. The method of claim 1, wherein the training corpus comprises at least one spectrum from a sample determined to be a pivot sample.

6. The method of claim 1, wherein the providing the nuclear magnetic resonance spectrum comprises providing nuclear magnetic resonance spectrum data points that cover a range of 10 ppm to 0.5 ppm, excluding a solute and any contaminant.

7. The method of claim 1, wherein the providing the nuclear magnetic resonance spectrum comprises providing nuclear magnetic resonance spectrum data points representing for at least regions for: lipid (0.9 ppm), BCAA, lipid (1.2 ppm), lipid (1.6 ppm), acetate, lipid (2.03 ppm), glutamine, lactate, glucose, myo-inositol, and betahydroxybutyrate.

8. The method of claim 1,
wherein the machine learning system is trained to output a classification of the nuclear magnetic resonance spectrum into one of a plurality of classes,
the method further comprising deriving a respective feature vector from the nuclear magnetic resonance spectrum for each pair of classes of the plurality of classes.

9. The method of claim 8, wherein each respective feature vector encodes differences between the nuclear magnetic resonance spectrum and a spectrum representing a respective base class, wherein the differences are determined at each of a plurality of spectral regions.

10. The method of claim 1, wherein the training corpus further comprises a group of benign disease biofluid nuclear magnetic resonance spectra.

11. A system for detecting a hypermetabolic cancer based on a nuclear magnetic resonance spectrum of a patient biofluid, the system comprising:
a machine learning system trained with a training corpus, the training corpus comprising a group of normal biofluid nuclear magnetic resonance spectra and a group of hypermetabolic cancer biofluid nuclear magnetic resonance spectra;
an electronic processor; and
a non-transitory computer-readable medium communicatively coupled to the electronic processor and comprising instructions that, when executed by the electronic processor, configure the electronic processor to perform actions comprising:
obtaining a nuclear magnetic resonance spectrum of a patient biofluid;
providing the nuclear magnetic resonance spectrum to the machine learning system; and
supplying an indication based on an output of the machine learning system, wherein the indication is representative of whether the nuclear magnetic resonance spectrum of the patient biofluid is indicative of cancer.

12. The system of claim 11, further comprising a nuclear magnetic resonance spectrometer, wherein the obtaining comprises obtaining the nuclear magnetic resonance spectrum of the patient biofluid from the nuclear magnetic resonance spectrometer.

13. The system of claim 11,
wherein the group of hypermetabolic cancer biofluid nuclear magnetic resonance spectra comprises pancreatic ductal adenocarcinoma biofluid nuclear magnetic resonance spectra, and
wherein the indication is representative of whether the nuclear magnetic resonance spectrum of the patient biofluid is indicative of pancreatic ductal adenocarcinoma.

14. The system of claim 11,
wherein the group of hypermetabolic cancer biofluid nuclear magnetic resonance spectra comprises non-small cell lung cancer biofluid nuclear magnetic resonance spectra, and
wherein the indication is representative of whether the nuclear magnetic resonance spectrum of the patient biofluid is indicative of non-small cell lung cancer.

15. The system of claim 11, wherein the training corpus comprises at least one spectrum from a sample determined to be a pivot sample.

16. The system of claim 11, wherein the providing the nuclear magnetic resonance spectrum comprises providing nuclear magnetic resonance spectrum data points that cover a range of 10 ppm to 0.5 ppm, excluding a solute and any contaminant.

17. The system of claim 11, wherein the providing the nuclear magnetic resonance spectrum comprises providing nuclear magnetic resonance spectrum data points representing for at least regions for: lipid (0.9 ppm), BCAA, lipid (1.2 ppm), lipid (1.6 ppm), acetate, lipid (2.03 ppm), glutamine, lactate, glucose, myo-inositol, and betahydroxybutyrate.

18. The system of claim 11,
wherein the machine learning system is trained to output a classification of the nuclear magnetic resonance spectrum into one of a plurality of classes, and
wherein the actions further comprise deriving a respective feature vector from the nuclear magnetic resonance spectrum for each pair of classes of the plurality of classes.

19. The system of claim 18, wherein each respective feature vector encodes differences between the nuclear magnetic resonance spectrum and a spectrum representing a respective base class, wherein the differences are determined at each of a plurality of spectral regions.

20. The system of claim 11, wherein the training corpus further comprises a group of benign disease biofluid nuclear magnetic resonance spectra.
